SEQUENCE LISTING (31 sequences)

SEQ ID NO: 1 - protein, 32 aa, Artificial Sequence
  Description of Artificial SequenceHIV
QQEKNEQELLELDKWASLWNWFDITNWLWYIK

SEQ ID NO: 2 - protein, 57 aa, Artificial Sequence
  Description of Artificial SequenceHIV
YKRWIILGLNKIVRMYSKQIINMWQEVGKAMYACTRPNYNKRKRIHIGPGRAFYTTK

SEQ ID NO: 3 - protein, 49 aa, Artificial Sequence
  Description of Artificial SequenceHIV
QQEKNEQELLELDKWASLWNWFNITNWLWYIKYKRWIILGLNKIVRMYS

SEQ ID NO: 4 - protein, 45 aa, Artificial Sequence
  Description of Artificial SequenceHIV
NEQELLELDKWASLWNWFNITNWLWYIKYKRWIILGLNKIVRMYS

SEQ ID NO: 5 - protein, 49 aa, Artificial Sequence
  Description of Artificial SequenceHIV
EAWLWDLLIWNLQFEWKNNWTEQNQLEKSYIKYKRWIILGLNKIVRMYS

SEQ ID NO: 6 - protein, 36 aa, Artificial Sequence
  Description of Artificial SequenceHIV
SLWNWFNITNWLWYIKGGGYKRWIILGLNKIVRMYS

SEQ ID NO: 7 - protein, 36 aa, Artificial Sequence
  Description of Artificial SequenceHIV
KNIWLSNYFWLINWWTGGGYKRWIILGLNKIVRMYS

SEQ ID NO: 8 - protein, 40 aa, Artificial Sequence
  Description of Artificial SequenceHIV
QQEKNEQELLELDKWASLWNGGGYKRWIILGLNKIVRMYS

SEQ ID NO: 9 - protein, 40 aa, Artificial Sequence
  Description of Artificial SequenceHIV
NKEQDQAEESLQLWEKLNWLGGGYKRWIILGLNKIVRMYS

SEQ ID NO: 10 - protein, 33 aa, Artificial Sequence
  Description of Artificial SequenceHIV
SLWNWFNITNWLWYIKYKRWIILGLNKIVRMYS

SEQ ID NO: 11 - protein, 33 aa, Artificial Sequence
  Description of Artificial SequenceHIV
KNIWLSNYFWLINWWTYKRWIILGLNKIVRMYS

SEQ ID NO: 12 - protein, 7 aa, Artificial Sequence
  Description of Artificial SequenceHIV
ELDKWAS

SEQ ID NO: 13 - protein, 37 aa, Artificial Sequence
  Description of Artificial SequenceHIV
QQEKNEQELLELDKWASLWNYKRWIILGLNKIVRMYS

SEQ ID NO: 14 - protein, 37 aa, Artificial Sequence
  Description of Artificial SequenceHIV
NKEQDQAEESLQLWEKLNWLYKRWIILGLNKIVRMYS

SEQ ID NO: 15 - protein, 117 aa, Artificial Sequence
  Description of Artificial SequenceHIV
MRVRGIQRNCQHLWRWGTLILGMLMICSAALGIWGCSGKLICTTAVPWNASWSNKSLEYTSLIHSLIEESQNQQEKNEQELLELDKWASLWNWFYKRWIILGLNKIVRMYSHHHHHH

SEQ ID NO: 16 - protein, 89 aa, Artificial Sequence
  Description of Artificial SequenceHIV
MRVRGIQRNCQHLWRWGTLILGMLMICSAAYTSLIHSLIEESQNQQEKNEQELLELDKWASLWNWFYKRWIILGLNKIVRMYSHHHHHH

SEQ ID NO: 17 - protein, 117 aa, Artificial Sequence
  Description of Artificial SequenceHIV
MRVRGIQRNCQHLWRWGTLILGMLMICSAAYKRWIILGLNKIVRMYSLGIWGCSGKLICTTAVPWNASWSNKSLEYTSLIHSLIEESQNQQEKNEQELLELDKWASLWNWFHHHHHH

SEQ ID NO: 18 - protein, 89 aa, Artificial Sequence
  Description of Artificial SequenceHIV
MRVRGIQRNCQHLWRWGTLILGMLMICSAAYKRWIILGLNKIVRMYSYTSLIHSLIEESQNQQEKNEQELLELDKWASLWNWFHHHHHH

SEQ ID NO: 19 - protein, 132 aa, Artificial Sequence
  Description of Artificial SequenceHIV
MRVRGIQRNCQHLWRWGTLILGMLMICSAALGIWGCSGKLICTTAVPWNASWSNKSLEYTSLIHSLIEESQNQQEKNEQELLELDKWASLWNWFNITNWLWYIKLFIMIVGGLVGLRIVFAVLSVVHHHHHH

SEQ ID NO: 20 - protein, 104 aa, Artificial Sequence
  Description of Artificial SequenceHIV
MRVRGIQRNCQHLWRWGTLILGMLMICSAAYTSLIHSLIEESQNQQEKNEQELLELDKWASLWNWFNITNWLWYIKLFIMIVGGLVGLRIVFAVLSVVHHHHHH

SEQ ID NO: 21 - protein, 7 aa, Artificial Sequence
  Description of Artificial SequenceHIV
WFNITNW

SEQ ID NO: 22 - protein, 6 aa, Artificial Sequence
  Description of Artificial SequenceHIV
NWFDIT

SEQ ID NO: 23 - protein, 6 aa, Artificial Sequence
  Description of Artificial SequenceHIV
ELDKWA

SEQ ID NO: 24 - protein, 852 aa, Artificial Sequence
  Description of Artificial SequenceHIV consensus sequence
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALL

SEQ ID NO: 25 - DNA, 2559 bp, Artificial Sequence
  Description of Artificial SequenceProbe
atgcgcgtgcgcggcatccagcgcaactgccagcacctgtggcgctggggcaccctgatcctgggcatgctgatgatctgctccgccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccaccaaggccaagcgccgcgtggtggagcgcgagaagcgcgccgtgggcatcggcgccgtgttcctgggcttcctgggcgccgccggctccaccatgggcgccgcctccatcaccctgaccgtgcaggcccgccagctgctgtccggcatcgtgcagcagcagtccaacctgctgcgcgccatcgaggcccagcagcacctgctgcagctgaccgtgtggggcatcaagcagctgcaggcccgcgtgctggccgtggagcgctacctgaaggaccagcagctgctgggcatctggggctgctccggcaagctgatctgcaccaccaccgtgccctggaactcctcctggtccaacaagtcccaggacgagatctgggacaacatgacctggatggagtgggagcgcgagatcaacaactacaccgacatcatctactccctgatcgaggagtcccagaaccagcaggagaagaacgagcaggagctgctggccctggacaagtgggcctccctgtggaactggttcgacatcaccaactggctgtggtacatcaagatcttcatcatgatcgtgggcggcctgatcggcctgcgcatcgtgttcgccgtgctgtccatcgtgaaccgcgtgcgccagggctactcccccctgtccttccagaccctgatccccaacccccgcggccccgaccgccccgagggcatcgaggaggagggcggcgagcaggaccgcgaccgctccatccgcctggtgaacggcttcctggccctggcctgggacgacctgcgctccctgtgcctgttctcctaccaccgcctgcgcgacttcatcctgatcgccgcccgcaccgtggagctgctgggccgcaagggcctgcgccgcggctgggaggccctgaagtacctgtggaacctgctgcagtactggggccaggagctgaagaactccgccatctccctgctggacaccaccgccatcgccgtggccgagggcaccgaccgcgtgatcgaggtggtgcagcgcgcctgccgcgccatcctgaacatcccccgccgcatccgccagggcctggagcgcgccctgctgtaa

SEQ ID NO: 26 - protein, 37 aa, Artificial Sequence
  Description of Artificial SequenceHIV
YKRWIILGLNKIVRMYSQQEKNEQELLELDKWASLWN

SEQ ID NO: 27 - protein, 33 aa, Artificial Sequence
  Description of Artificial SequenceHIV
YKRWIILGLNKIVRMYSSLWNWFNITNWLWYIK

SEQ ID NO: 28 - DNA, 22 bp, Artificial Sequence
  Description of Artificial SequenceProbe
tcgtcgttgtcgttttgtcgtt

SEQ ID NO: 29 - protein, 28 aa, Artificial Sequence
  Description of Artificial SequenceHIV
NEQELLELDKWASLWNWFNITNWLWYIK

SEQ ID NO: 30 - protein, 23 aa, Artificial Sequence
  Description of Artificial SequenceHIV
FIMIVGGLVGLRIVFAVLSIVNR

SEQ ID NO: 31 - protein, 32 aa, Artificial Sequence
  Description of Artificial SequenceHIV
KWASLWNWFDITNWLYKRWIILGLNKIVRMYS

(19) United States
(12) Patent Application Publication
(10) Pub. No.: US 2014/0322262 A1
(43) Pub. Date: Oct. 30, 2014

(54) METHOD OF INDUCING NEUTRALIZING ANTIBODIES TO HUMAN IMMUNODEFICIENCY VIRUS

(71) Applicants: Leonard D. Spicer, Durham, NC (US); Patrick N. Reardon, Durham, NC (US); Barton F. Haynes, Durham, NC (US); S. Munir Alam, Durham, NC (US)

(72) Inventors: Leonard D. Spicer, Durham, NC (US); Patrick N. Reardon, Durham, NC (US); Barton F. Haynes, Durham, NC (US); S. Munir Alam, Durham, NC (US)

(73) Assignee: Duke University, Durham (NC)

(21) Appl. No.: 14/110,490

(22) PCT Filed: Apr. 9, 2012
(86) PCT No.: PCT/US12/32717

§ 371 Date: Jul. 17, 2014

Related U.S. Application Data
(63) Continuation-in-part of application No. 13/083,466, filed on Apr. 8, 2011.
Application No. 13/083,466 is a continuation-in-part of application No. 12/450,779, filed on Oct. 13, 2009.
(60) Provisional application No. 60/960,413, filed on Sep. 28, 2007.

(51) Int. Cl. A61K 39/385; A61K 39/21

(52) U.S. Cl. CPC A61K 39/385; A61K 39/21
USPC 424/196.11; 530/350

(57) ABSTRACT

The present invention relates, in general, to human immunodeficiency virus (HIV), and, in particular, to a method of inducing neutralizing antibodies to HIV and to compounds and compositions suitable for use in such a method.

This application claims priority from U.S. application Ser. No. 13/083,466, filed Apr. 8, 2011, which is a continuation-in-part of U.S. application Ser. No. 12/450,779, filed Oct. 13, 2009, which is the U.S. national phase of International Application No. PCT/US2008/004709, filed Apr. 11, 2008 which designated the U.S. and claims priority to U.S. application Ser. No. 11/785,077, filed Apr. 13, 2007, U.S. application Ser. No. 11/812,992, filed Jun. 22, 2007 and U.S. Provisional Application No. 60/960,413, filed Sep. 28, 2007, the entire contents of these application are incorporated herein by reference

This invention was made with government support under Grant Number U01 AI 067854 awarded by the National Institutes of Health. The government has certain rights in the invention

### TECHNICAL FIELD

The present invention relates, in general, to human immunodeficiency virus (HIV), and, in particular, to a method of inducing neutralizing antibodies to HIV and to compounds and compositions suitable for use in such a method.

### BACKGROUND

The first antibodies that are made in acute HIV-1 infection are against the CD4 binding site (Moore et al, J. Virol. 68(8) 5142 (1994)), the CCR5 co-receptor binding site (Choe et al, Cell 114(2):161-170 (2003)), and the V3 loop (Moore et al, J. Acquir. Immun. Def. Syn. 7(4):332 (1994)). However, these antibodies do not control HIV-1 and are easily escaped (Burton et al, Nature Immun. 5:233-236 (2004), Wei et al, Nature 422(6929):307-312 (2003)). Neutralizing antibodies against autologous virus develop fifty to sixty days after infection, but antibodies capable of neutralizing heterologous HIV-1 strains do not arise until after the first year of infection (Richman et al, Proc. Natl. Acad. Sci. USA 100(7):4144-4149 (2003), Wei et al, Nature 422(6929):307-312 (2003)).

The four epitopes on HIV-1 envelope to which rare broadly reactive neutralizing antibodies bind are the CD4 binding site (CD4BS) (mab (monoclonal antibody) IgG1b12) (Zwick et al, J. Virol. 77(10):5863-5876 (2003)), the membrane proximal external region (MPER) epitopes defined by human mabs 2F5 and 4E10 (Armbruster et al, J. Antimicrob. Chemother. 54:915-920 (2004), Stiegler and Katinger, J. Antimicrob. Chemother. 51:757-759 (2003), Zwick et al, Journal of Virology 79:1252-1261 (2005), Purtscher et al, AIDS 10:587 (1996)) (FIG. 1), and the mannan glycan epitope defined by human mab 2G12 (Scanlan et al, Adv. Exper. Med. Biol. 535:205-218 (2003)). These four rare human mabs are all unusual: two are IgG3 (2F5 and 4E10), one has a unique Ig dimer structure (2G12), one has a very hydrophobic CDR3 (2F5) (Ofek et al, J. Virol. 198:10724 (2004)), and, in all four, the CDR3 is unusually long (Burton et al, Nature Immunol. 5(3):233-236 (2004), Kunert et al, AIDS Res. Hum. Retroviruses 20(7):755-762 (2004), Zwick et al, J. Virol. 78(6):3155-3161 (2004), Cardoso et al, Immunity 22:163-172 (2005)). Of these, 2F5- and 4E10-like human mabs are quite rare. Acute HIV-1 patients do not make antibodies against the MPER or 2G12 epitopes (Robinson, unpublished (2005), Shaw, unpublished (2005), MPER can be defined as amino acids 652 to 683 of HIV envelope (Cardoso et al, Immunity 22:163-173 (2005) (e.g., QQEKNEQELLELDKWASLWNWFDITNWLWYIK). CD4 binding site (BS) antibodies are commonly made early in HIV-1 infection, but these antibodies generally do not have the broad spectrum of neutralization shown by mab IgG1b12 (Burton et al, Nat. Immunol. 5(3):233-236 (2004)).

A number of epitopes of the HIV-1 envelope have been shown to cross-react with host tissues (Pinto et al, AIDS Res. Hum. Retrov. 10:823-828 (1994), Douvas et al, AIDS Res. Hum. Retrov. 10:253-262 (1994), Douvas et al, AIDS Res. Hum. Retrov. 12:1509-1517 (1996)), and autoimmune patients have been shown to make antibodies that cross-react with HIV proteins (Pinto et al, AIDS Res. Hum. Retrov. 10:823-828 (1994), Douvas et al, AIDS Res. Hum. Retrov. 10:253-262 (1994), Douvas et al, AIDS Res. Hum. Retrov. 12:1509-1517 (1996), Barthel et al, Semin. Arthr. Rheum. 23:1-7 (1993)). Similarly, induction of immune responses to self-epitopes has been suggested to be a cause of the autoimmune abnormalities and T cell depletion in AIDS (Douvas et al, AIDS Res. Hum. Retrov. 12:1509-1517 (1996), Ziegler et al, Clin. Immunol. Immunopath. 41:305-313 (1986)).

High affinity peptide ligands for the 2F5 mab have been made that induce high levels of antibody against the peptide but do not broadly neutralize HIV-1 primary isolates (McGaughey et al, Biochemistry 42(11):3214-3223 (2003), Zhang et al, J. Virol. 78(15):8342-8348 (2004), rev. in Zwick et al, J. Virol. 79:1252-1261 (2005)). These results have been interpreted to mean that the peptide ligands for 2F5 are not in the appropriate conformation for induction of anti-MPER antibodies (Burton et al, Nature Immunology 5(3):233-236 (2004), Zwick et al, J. Virol. 79:1252-1261 (2005)). A series of highly constrained HIV-1 Env immunogens have been made with the IgG1b12, 2G12, 2F5 and 4E10 epitopes stably expressed, and it has been demonstrated that these immunogens do not induce broadly reactive neutralizing antibodies in guinea pigs or rabbits, and, specifically, do not make neutralizing antibodies to the MPER epitopes (Liao et al, J. Virol. 78(10):5270-5278 (2004); Haynes, unpublished (2005)). These results have raised the question as to whether broadly reactive neutralizing antibodies to HIV-1 envelope are not made in normal animals and humans because they cannot be made.

Because long, hydrophobic CDR3 regions are typical of natural polyreactive autoantibodies (Meffre et al, J. Clin. Invest. 108:879-886 (2001), Ramsland et al, Exp. Clin. Immun. 18:176-198 (2001)), and HIV-1-infected patient B lymphocytes are polyclonally driven to make cardiolipin antibodies (Weiss et al, Clin. Immunol. Immunopathol. 77:69-74 (1995), Grunewald et al, Clin. Exp. Immunol. 15:464-71 (1999)), studies were undertaken to assay these and other anti-HIV-1 mabs for cardiolipin and other autoantigen reactivities. The present invention results, at least in part, from the realization that two broadly reactive HIV-1 envelope gp 41 human mabs, 2F5 and 4E10, are polyspecific autoantibodies reactive with cardiolipin.

The invention relates, in part, to MPER peptide-liposome conjugates and to methods of using same to induce broadly neutralizing gp41 MPER antibodies. The invention provides, in one embodiment, a molecular conjugate that presents the MPER of gp41 as a trimer attached to biological membrane mimetics.

### SUMMARY OF THE INVENTION

The present invention relates generally to human HIV. More specifically, the invention relates to a method of inducing neutralizing antibodies to HIV and to compounds and compositions suitable for use in such a method. In a specific embodiment, the present invention provides immunogens that present MPER epitopes in their native membrane bound environment, and immunization methods using such immunogens that break tolerance.

Objects and advantages of the present invention will be clear from the description that follows.

### BRIEF DESCRIPTION OF THE DRAWINGS

FIG. 1. Broadly neutralizing antibodies (2F5, 4E10) bind to epitopes that lie proximal to the host membrane. Both 2F5 and 4E1 mAbs are IgG3, have long CDR3s, and bind to epitopes that lie within HIV-1 gp41 (aa 660-683) membrane proximal external region (MPER).

FIGS. 2A-2D. Reactivity of 2F5, 4E10, IgG1b12 Mabs with human Hep-2 epithelial cells. FIG. 2A shows Mab 2F5 reacting with Hep-2 cells in a diffuse cytoplasmic and nuclear pattern, FIG. 2B shows Mab 4E10 reacting with HEp-2 cells in a pattern similar to 2F5. FIG. 2C shows Mab IgG1b12 reacting with Hep-2 cells in a diffuse cytoplasmic pattern, with nucleoli reactive in the nucleus. FIG. 2C insert shows higher magnification of cells showing the nucleolar reactivity of IgG1b12 (arrows). FIG. 2D shows negative reactivity of Mab 1.9F on Hep-2 cells. Antibody amounts per slide assayed in FIGS. 2A-2D were 3.75 μg per slide of Mab. Mab 2F5 was positive on HEp-2 cells at 0.125 μg per slide (5 μg/ml). Mab 4E10 was positive on HEp-2 at 0.125 μg per slide (5 μg/ml), and IgG1b12 was positive at 1.25 μg per slide (50 μg/ml). All Figs. X200.; FIG. 2C insert X400. Images shown are from an experiment representative of three performed.

FIGS. 3A-3D. Assay of Mabs 2F5 and 4E10 against lipids and specificity of binding. FIG. 3A shows ELISA reactivity of MAbs 4E10 (solid bars) and 2F5 (open bars) to cardiolipin (CL), phosphatidylserine (PS), phosphatidylcholine (PC), phophatidylethanolamine (PE), and sphingomyelin (SM). Whereas both 4E10 and 2F5 reacted with cardiolipin, only 4E10 reacted with the other lipids tested. Reactivity of control human anti-CCR5 binding site MAb 1.7b was negative (data not shown). Reactivity of MAbs against empty coated plate was similarly negative (not shown). To show specificity of binding of MAb 2F5 to cardiolipin, 150-300 μg/ml of 2F5 and 1000 μg/ml of anti-2F5 idiotype murine MAb 3H6, which blocks the neutralization of HIV-1 by MAb 2F5 (Kunert et al, AIDS 16:667 (2002)), were used. The 2F5 anti-idiotype significantly blocked the binding of MAb 2F5 to cardiolipin by a mean of 70% in 3 separate experiments (p<0.03) (FIG. 3B). In a separate ELISA, MAb 2F5 bound to cardiolipin in half-maximal (EC50) response of 660 nM (not shown). FIG. 3C shows the dose response curve of 4E10 MAb binding to cardiolipin. The half-maximal (EC50) response of 4E10 binding (80 nM) was calculated from a four parametric, sigmoidal curve fitting analysis. Binding data was acquired from an ELISA of 4E10 MAb binding (0.5 nM-1000 nM) to cardiolipin coated on ELISA plate (1.35 μg/well). FIG. 3D shows soluble HIV-1 Env gp140 oligomers (CON-S) expressing the 4E10 epitope inhibits binding of 4E10 MAb to cardiolipin. The IC50 of inhibition of 4E10 binding to cardiolipin was calculated to be 145 nM. The inhibition assay was carried out by using varying concentrations of gp140 (19.25-1230 nM) mixed with 10 μg/ml of 4E10 MAb, which were then added to wells containing 1.35 μg of cardiolipin. MAb 3H6 (1 mg/ml) (but not control MAb) also blocked the binding of MAb 2F5 to SSA/Ro, centromere B, and histones (not shown). All data in FIGS. 3A-3D are representative of at least two experiments performed.

FIGS. 4A and 4B. Amino acid (FIG. 4A) and nucleic acid (FIG. 4B) sequences of CON-S Env gp160. A CR form of the protein of FIG. 4A was used in Example 2. (Gp140CFI refers to an HIV-1 envelope design with the cleavage site (C), fusion site (F), and gp41 immunodominant region (I) deleted in addition to the deletion of the transmembrane and cytoplasmic domains.)

FIG. 5. Structures of phosphospholipids used in immunization regimens and resulting neutralization titers.

FIGS. 6A and 6B. Peptide sequences used in the generation of peptide-liposome conjugates. The nominal epitopes of mAbs 2F5 and 4E10 binding epitopes include sequences ELDKWAS and WFNITNW, respectively, and are underlined. The V3 sequences were derived from gp120 of HIV-1 MN strain and were used as a control construct. Scrambled sequences are used controls.

FIG. 7. Schematic presentation of various designs of MPER gp41 constructs. The functional regions are indicated above the schematic constructs. Amino acid sequences are indicated below each of schematic constructs. Initiation and maturation signal sequences are highlighted in blue; immunodominant regions are highlighted in bold; MPER regions are highlighted in brown and GTH1 domains are highlighted in red and transmembrane domains are underlined. His-tags were added to the C-terminal ends of the constructs for easy purification and are highlighted in green.

FIG. 8. Binding of mAb 4E10 to peptide-liposome conjugates. BIAcore binding curves show specific and markedly higher binding of mAb 4E10 to GTH1-4E10 liposomes. Low levels of binding with fast kinetics to GTH1-2F5 liposomes were also detected.

FIG. 9. Binding of 2F5 mAb to peptide-liposomes. MAb 2F5 bound specifically to GTH1-2F5 liposomes and showed no binding to GTH1-4E10 liposomes.

FIG. 10. A32 mAb binding to peptide-liposomes. A control anti-gp120 Mab, A32, showed no binding to any of the liposome conjugates. 17b, a CD4-inducible mAb, also showed no binding to the above liposome conjugates (data not shown).

FIG. 11. Generation of fluorescein conjugated peptide-liposomes. Peptide-liposomes were conjugated with a fluorescein tag by incorporating fluorescein-POPE in the lipid composition. Binding assays show that the specificity of mAb 4E10 binding is retained in fluorescein conjugated liposomes. Fluorescein-conjugated GTH1-2F5 liposomes gave similar results.

FIG. 12. Reactivity of immunized guinea pig sera with 4E10 peptide. ELISA binding assay show strong positive reactivity of sera to 4E10 peptide from two guinea pigs immunized with GTH1-4E10 liposomes. All pre-bleed sera gave background binding while a low level of binding was observed in a serum from an animal immunized with 4E10 peptide. Both the positive sera from the peptide-liposome immunized animals also showed neutralizing activity (Table 2). One serum (1102) showed neutralization of MN and SS1196 strains with antibody titers at 1:209 and 1:32 respectively. The second serum (1103) was only effective against the MN virus (1:60).

FIG. 13. MPER mAb binding to peptide epitope follows a simple model (Langmuir equation).

FIG. 14. Neutralizing MPER mAb binding to epitope peptide-lipid conjugate follows a 2-step conformational change model.

FIG. 15. Human cluster II mAbs (98-6, 167-D, 126-6) bind strongly to Env gp140.

FIGS. 16A-16D. Human Cluster II mAbs bound strongly to the anionic phospholipid, cardiolipin.

FIGS. 17A-17E. Human Cluster II mAb 98-6 bound to 2F5 peptide-lipid conjugates with higher avidity and followed the 2-step conformational change model.

FIGS. 18A-18C: Structures of TLR adjuvants formulated with liposomes. FIG. 18A Lipid A; FIG. 18B Oligo CpG; FIG. 18C R-848.

FIGS. 19A-19C: Pictorial representation of TLR adjuvant-MPER peptide liposomes. FIG. 19A Lipid A; FIG. 19B Oligo CpG; FIG. 19C R-848.

FIGS. 20A-20C: Interaction of 2F5 mAB with MPER peptide-liposomes conjugated to TLR adjuvants. FIG. 20A shows strong binding of 2F5 mab to gp41 MPER liposome constructs with Lipid A (200 μg dose equivalent). FIG. 20B shows binding of 2F5 mAb to oCpG (50 μg dose equivalent) conjugated gp41 MPER liposomes. FIG. 20C shows binding of 2F5 mAb to R848-conjugated gp41 MPER containing liposomes. In comparison to control liposomes with only TLR adjuvants, strong binding of 2F5 mAb was observed to each of the gp41 MPER-adjuvant liposomal constructs.

FIG. 21: Amino acid sequence of the MPER656-TMD peptide.

FIGS. 22A and 22B: Pictorial representation of liposome immobilization on L-1 chip. FIG. 22A Synthetic liposomes. FIG. 22B MPER656-TMD liposomes.

FIGS. 23A and 23B: Interaction of 2F5 and 4E10 mAbs with MPER656-TMD liposomes. FIG. 23A 2F5 and FIG. 23B 4E10.

FIG. 24. Schematic representation of the FMS peptide.

FIG. 25. Schematic representation of fusion protein used to express the FMS peptide.

FIG. 26. Representative plot of equilibrium analytical ultracentrifugation data to a monomer to trimer equilibrium model.

FIG. 27. 2F5 binding to FMS conjugate.

FIGS. 28A and 28B: Average structures of the two prominent conformers of MPER trimer conjugated to a micelle; ribbon and space filling representations. (FIG. 28A) First conformer where blue indicates core residues in the 2F5 epitope and red indicates core residues in the 4E10 epitope. (FIG. 28B) Second conformer with some color coding.

FIG. 29: Enlargement of the 2F5 epitope region of the trimer showing the positions of the D664, K665 and W666 side chains.

FIGS. 30A and 30B. FIG. 30A. Schematic of extended construct. FIG. 30B. SDS-PAGE gel of purified extended construct (lane 1).

FIG. 31. HN-HSQC NMR spectrum of gp41 construct shown in FIG. 30A. Data were acquired on a Varian 800 MHz spectrometer.

FIG. 32. Preliminary binding data. DMPC liposomes conjugated to gp41 trimer construct were assayed for 2F5 binding (blue—upper curve) and 4E10 binding (red—lower curve). The curves show that the construct binds both neutralizing antibodies.

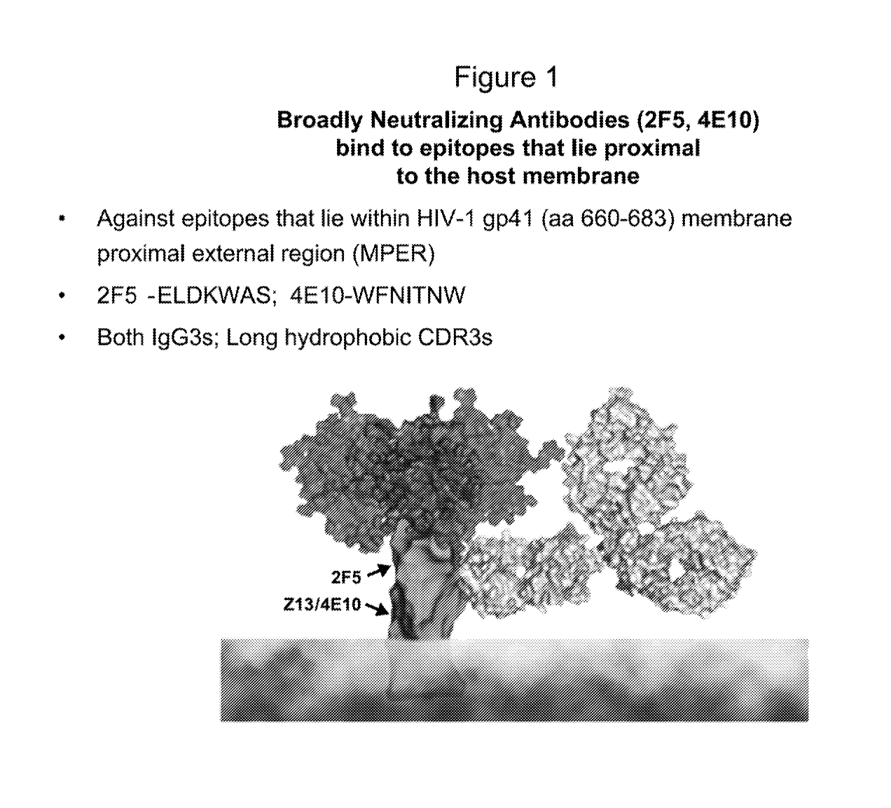

### DETAILED DESCRIPTION OF THE INVENTION

The present invention results, at least in part, from studies demonstrating that certain broadly neutralizing HIV-1 antibodies are autoantibodies. A large number of HIV⁺ patients transiently make low levels of such antibodies, however, the studies described herein indicate that gp41 epitopes do not induce these antibody specificities but, rather, that cross-reactive autoantigens, including cardiolipin, are the priming antigen.

The present invention provides a method of inducing antibodies that neutralize HIV. The method comprises administering to a patient in need thereof an amount of at least one heterologous (e.g., non-human) or homologous (e.g., human) cross-reactive autoantigen sufficient to effect the induction. Cross-reactive autoantigens suitable for use in the instant invention include cardiolipin, SS-A/RO, dsDNA from bacteria or mammalian cells, centromere B protein and RiBo nucleoprotein (RNP).

Suitable autoantigens also include phospholipids in addition to cardiolipin, such as phosphatidylserine, phosphatidylethanolamine, phosphatidylcholine, phosphotidylinositol, sphingomyelin, and derivatives thereof, e.g., 1-palmitoyl-2-oleoyl-sn-glycero-3-[phospho-L-serine] (POPS), 1-palmitoyl-2-oleoyl-phosphatidylethanolamine (POPE), and dioleoyl phosphatidylethanolamine (DOPE). Use of hexagonal II phases of phospholipids can be advantageous and phospholipids that readily form hexagonally packed cylinders of the hexagonal II tubular phase (e.g., under physiological conditions) are preferred, as are phospholipids that can be stabilized in the hexagonal II phase. (See Rauch et al, Proc. Natl. Acad. Sci. USA 87:4112-4114 (1990); Aguilar et al et al, J. Biol. Chem. 274: 25193-25196 (1999)).

Fragments of such autoantigens comprising the cross-reactive epitopes can also be used.

The autoantigen, or fragment thereof, can be used, for example, in prime boost regimens that can be readily optimized by one skilled in the art (DNA sequences encoding proteinaceous components of such regimens can be administered under conditions such that the proteinaceous component is produced in vivo). By way of example, cross-reactive autoantigen can be used as a first vaccine prime to boost natural auto-antibodies (e.g., anti-cardiolipin 4E10- and 2F5-like antibodies). Either autoantigen (e.g., cardiolipin (or fragment thereof)), or an HIV-envelope protein/polypeptide/peptide comprising a cross-reactive epitope(s), such as the 2F5 and/or 4E10 epitopes (which epitopes can include at least the sequences ELDKWA and NWFDIT, respectively), can be used as the boost. (See sequences disclosed in PCT/US04/30397.) (It will be appreciated that HIV-envelope is not an autoantigen.)

The mode of administration of the autoantigen and/or HIV-protein/polypeptide/peptide, or encoding sequence, can vary with the immunogen, the patient and the effect sought, similarly, the dose administered. Optimum dosage regimens can be readily determined by one skilled in the art. Typically, administration is subcutaneous, intramuscular, intravenous, intranasal or oral.

The immunogenic agents can be administered in combination with an adjuvant. While a variety of adjuvants can be used, preferred adjuvants include CpG oligonucleotides and other agents (e.g., TRL9 agonists) that can break tolerance to autoantigens without inducing autoimmune disease (Tran et al, Clin. Immunol. 109:278-287 (2003), US Appin Nos. 20030181406, 20040006242, 20040006032, 20040092472, 20040067905, 20040053880, 20040152649, 20040171086, 20040198680, 200500059619).

In a specific embodiment, the invention relates to a liposome based adjuvant conjugate that presents Toll like receptor (TLR) ligands and HIV-1 gp41 neutralizing antigens. In accordance with this embodiment, immune response enhancing TLR ligands such as Lipid A, oligo CpG and R-848 can be formulated individually into liposomes that have HIV-1 gp41 MPER peptide immunogen conjugated in them. As described in Example 7 below, broadly neutralizing gp41 membrane proximal external region (MPER) antibodies (2F5, 4E10) bind strongly to each of the TLR ligand adjuvant associated liposome constructs. Constructs of this embodiment have application in enhancing an immune response against poorly immunogenic of HIV-1 gp41 MPER.

In a further specific embodiment, the present invention relates to the transmembrane domain anchoring of HIV-1 gp41 MPER peptide to liposomes for functional display of the epitopes of broadly neutralizing antibodies, such as 2F5 and 4E10. In accordance with this embodiment, the transmembrane domain (TMD) of HIV-1 gp41 can be used to anchor the MPER peptide into liposomes comprising synthetic lipids. As described in Example 8 below, broadly neutralizing anti-gp41 antibodies 2F5 and 4E10 both bind to the MPER-TMD-liposome conjugates. This construct provides a strategy to present gp41 neutralizing epitopes anchored on liposome using the native TMD of HIV-1. Induction of trimerization of the TMD can facilitate formation of trimeric forms of gp41 MPER.

Described in Example 9 below is a molecular conjugate that presents the MPER of HIV-1 gp41 as a trimer attached to a biological membrane mimetic (e.g., a phospholipid membrane). The trimeric MPER construct described uses the foldon domain from T4 fibritin to trimerize the N-terminus of the MPER while allowing the C-terminus to freely associate with itself and/or the phospholipid membrane. While foldon was used in the study described, other trimerization domains, such as GCN4, could also be used. No trimerization is imposed on the C-terminal region by additional sequences. A flexible linker (e.g., a GSSG or other peptide linker, or other flexible segment that allows conformational flexibility) can be incorporated between the C-terminus of the foldon and the N-terminus of the MPER, for example, to prevent the foldon structure from influencing the MPER trimer structure.

The results provided in Example 9 show that the construct is trimeric in dodecylphosphocholine (DPC) detergent micelles. The trimer construct binds one micelle of DPC detergent, based upon the aggregation number of DPC—this yields a total molecular weight of the detergent-protein complex of approximately 42 kD. SPR analysis shows specific binding to this MPER trimer by the broadly neutralizing antibodies 2F5 and 4E10 when displayed on DMPC liposomes. The observed Kd's on DMPC liposomes are 0.18 nM and 27 nM, respectively. Antibodies 2F5 and 4E10 also bind specifically to the construct in the DPC detergent micelles used for ultracentrifugation and subsequent NMR analysis.

The MPER trimer/phospholipid conjugate described in Example 9 gives good multidimensional NMR spectra in DPC detergent micelles, making it possible to assign and structurally characterize the MPER trimer. Based on NOE distance constraints, dihedral angles derived from observed backbone chemical shift data, and residual dipolar couplings in stretched polyacrylamide gels, it was possible to calculate the structure of each of the identical monomer subunits and the trimer structure when bound to the membrane. It is clear from the subunit structures that the MPER adopts a helical conformation. There is evidence for a slight bend in the helix at residues W672 and F673. Initial dynamics studies using heteronuclear NOE's show that the flexible linker region is dynamic, as expected. Additionally, there is evidence for increased flexibility associated with the region where the bend in the helix has been observed.

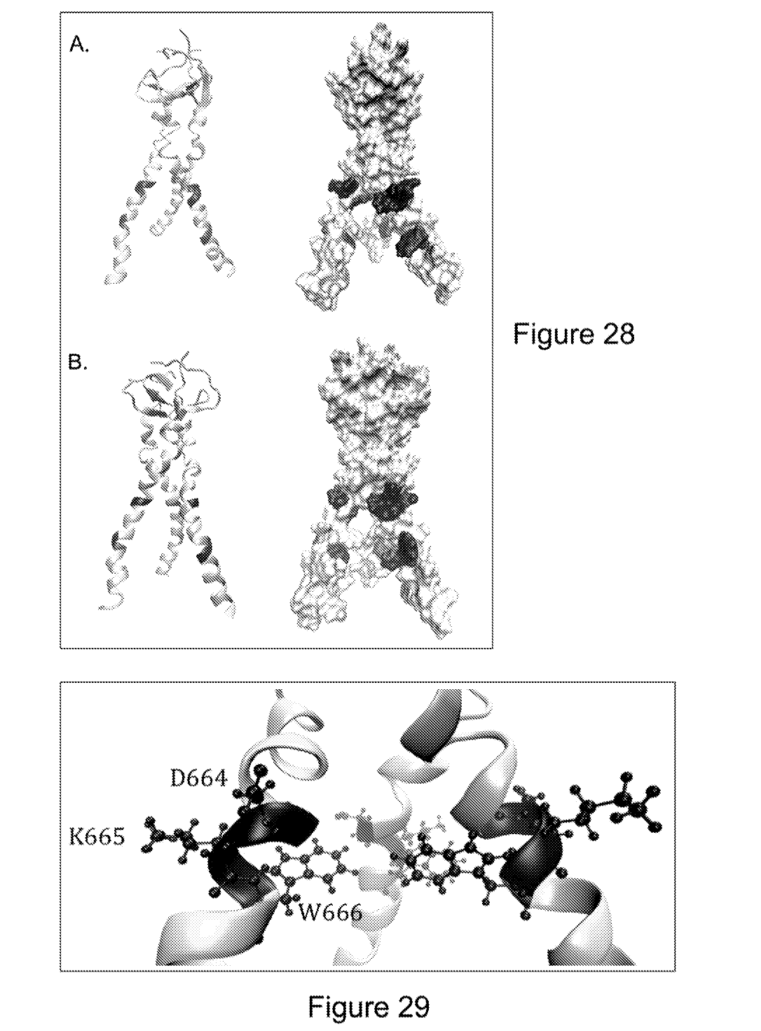

To construct the trimer structure from the well characterized subunits, additional NMR data were obtained from a differentially isotope labeled trimer. This enables the detection of intermolecular distance restraints. Initial models generated with preliminary data of this type show that the N-terminal region of the MPER is a relatively tight helical trimer, while the C-terminal region is more splayed, as illustrated in FIG. 28. There appear to be two low energy conformations for the MPER trimer in solution, and both present the 2F5 and 4E10 epitopes for antibody binding. These structures indicate that D664 and K665 of the 2F5 epitope are fully exposed, while W666 is interacting in the trimer interface but also partially accessible as illustrated by the enlarged view in FIG. 29.

Described in Example 10 is an extended molecular construct fora trimeric gp41 domain that contains the MPER. This extended construct includes the HIV-1 HR-2 gp41 domain. The HR-2 domain plays a critical role in viral infection when it re-folds with HR-1 to form the six-helix bundle that drives membrane fusion. This construct presents the lipid associated MPER and HR-2 domains in a native like pre-fusion intermediate state.

The invention includes compositions suitable for use in the instant method, including compositions comprising the autoantigen, and/or HIV protein/polypeptide/peptide comprising one or more cross-reactive epitopes (e.g., 4E10 and/or 2F5 epitopes), or 4E10 or 2F5 epitope mimics, and a carrier. When a DNA prime or boost can be used, suitable formulations include a DNA prime and a recombinant adenovirus boost and a DNA prime and a recombinant mycobacteria boost, where the DNA or the vectors encode, for example, either HIV envelope or a protein autoantigen, such as SS-A/Ro. Other combinations of these vectors can be used as primes or boosts, either with or without HIV protein/polypeptide/peptide and/or autoantigen. The composition can be present, for example, in a form suitable for injection or nasal administration. Advantageously, the composition is sterile. The composition can be present in dosage unit form.

The present invention also relates to a passive immunotherapy approach wherein B cells from patients with a primary autoimmune disease, such as systemic lupus erythematosis (SLE) or anti-phospholipid antibody syndrome or patients with infectious diseases such as syphilis, leishmaniasis, and leprosy, can be used in the production of cross-reactive antibodies (including monoclonal antibodies other than 4E10 and 2F5). Autoimmune disease patients can make antibodies that, in some capacity, have the ability to neutralize HIV-1, either in binding to the HIV envelope or in binding to lipids on the surface of the virion, or both. Moreover autoimmune disease patients can make a protective neutralizing type antibody either constitutively or after HIV-1 infection.

That is, the invention includes the use of B cells from SLE patients, as well as other patients with disordered immunoregulation (that is, patients with a primary autoimmune disease, or a non-HIV infection such as those noted above, that produce autoantibodies cross-reactive with HIV envelope), in the production of immortal cell lines that provide a source of antibodies that cross-react with HIV envelope (such as 2F5-like and 4E10-like antibodies) (see Stiegler et al, AIDS Res. Hum. Retroviruses 17:1757-1765 (2001), Armbruster et al, J. Antimicrob. Chemother. 54:915-920 (2004), U.S. Pat. No. 5,831,034). Advantageously, the B cells are from an SLE patient (or patient with another primary autoimmune disease) that is HIV infected or that has received an envelope-based HIV vaccine (while not wishing to be bound by theory, HIV infection or vaccination may serve to “boost” primed B1 cells (e.g., cardiolipin-primed B1 cells) to produce 2F5- and/or 4E10-like antibodies and escape deletion (which would occur in a normal subject)—the “boost” may trigger somatic hypermutation so that the resulting Ig genes encode antibodies that fit 2F5 and or 4E10-like epitopes—or that fit other gp160 epitopes that induce broadly neutralizing antibodies but are deleted in normal subjects). The production of immortal cell lines from B cells can be effected using any of a variety of art recognized techniques, including, but not limited to, fusing such B cells with myeloma cells to produce hybridomas. The invention also includes antibodies from normal subjects and from autoimmune disease patients that do not react HIV envelope but rather with virus-infected cells and or virions, that is, they bind to lipid on the virus or virus-infected cells (see Example 6).

Once selected, sequences encoding such cross-reactive antibodies (or binding fragments thereof) can be cloned and amplified (see, for example, Huse et al, Science 246:1275-1281 (1989), and phage-display technology as described in WO 91/17271, WO 92/01047, U.S. Pat. Nos. 5,877,218, 5,871,907, 5,858,657, 5,837,242, 5,733,743 and 5,565,332). Soluble antibodies for therapy can then be designed and produced using art recognized techniques (Stiegler et al, AIDS Res. Hum. Retroviruses 17:1757-1765 (2001), Armbruster et al, J. Antimicrob. Chemother. 54:915-920 (2004)). Suitable antibodies can be produced in Chinese Hamster Ovary (CHO) cells.

In accordance with this approach, the antibody (or binding fragment thereof) can be administered in doses ranging from about 10 to 100 mg/dose, preferably 25 mg/dose. The dosage and frequency can vary with the antibody (or binding fragment thereof), the patient and the effect sought (see Armbruster et al, J. Antimicrob. Chemother. 54:915-920 (2004)). The antibodies described above can be used prophylactically or therapeutically.

The antibodies (or binding fragments thereof), or DNA encoding the antibodies or binding fragments, can be formulated with a carrier (e.g., pharmaceutically acceptable carrier) and can be administered by, for example, parenteral, intravenous, subcutaneous, intramuscular or intranasal routes.

Finally, animal species such as camels (Ramsland et al, Exp. Clin. Immunogenet. 18:176-198 (2001), Litman et al, Annu. Rev. Immunol. 7:109-147 (1999)), cows (Ramsland et al, Exp. Clin. Immunogenet. 18:176-198 (2001), Litman et al, Annu. Rev. Immunol. 7:109-147 (1999)) and sharks (Ramsland et al, Exp. Clin. Immunogenet. 18:176-198 (2001), Litman et al, Annu. Rev. Immunol. 7:109-147 (1999), Hohman et al, Proc. Natl. Acad. Sci. USA. 90:9882-9886 (1993)) have very long CDR3 lengths, and their antibodies show polyreactivitiy. These engineered CDR3s that show polyreactivity to HIV envelope can be utilized for making potent therapeutic antibodies (e.g, monoclonal antibodies, including, for example, chimeric and humanized antibodies, and antigen binding fragments thereof) to HIV and to many infectious agents.

In a specific embodiment, the present invention further relates to synthetic liposome-peptide conjugates and to methods of using same as immunogens for the generation of broadly neutralizing antibodies against HIV-1. This embodiment of the invention provides compositions and methods for embedding into synthetic liposomes nominal epitope peptides of broadly neutralizing antibodies that bind to the MPER of HIV-1 gp41. Also provided are immunization strategies and protocols for the generation of anti-HIV-1 neutralizing antibodies and for the detection of antigen specific B cell responses.

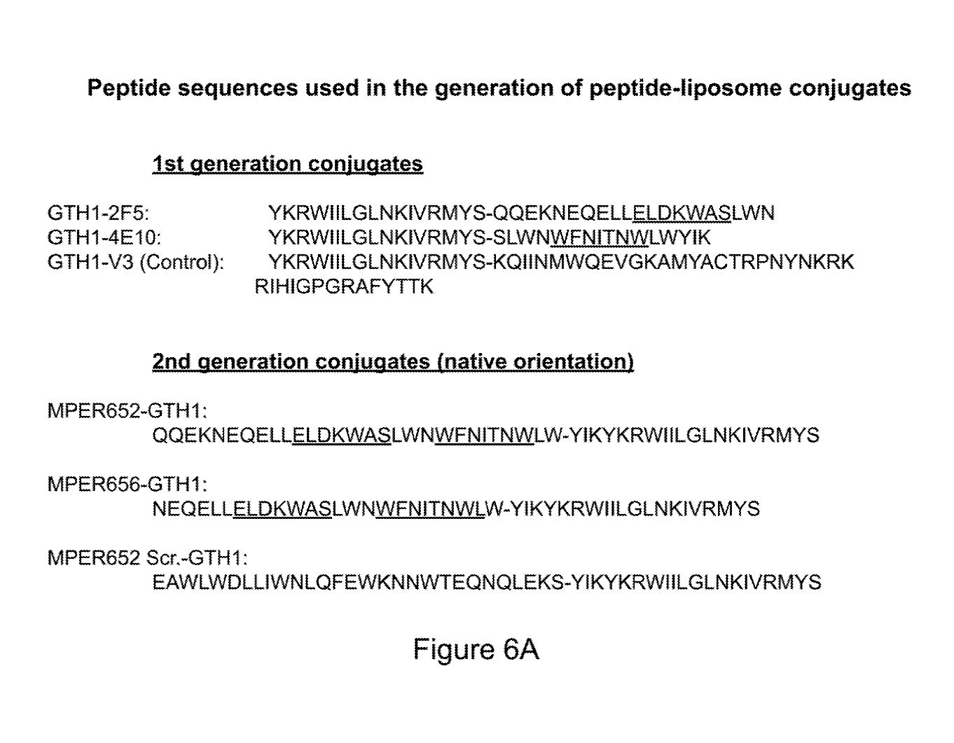

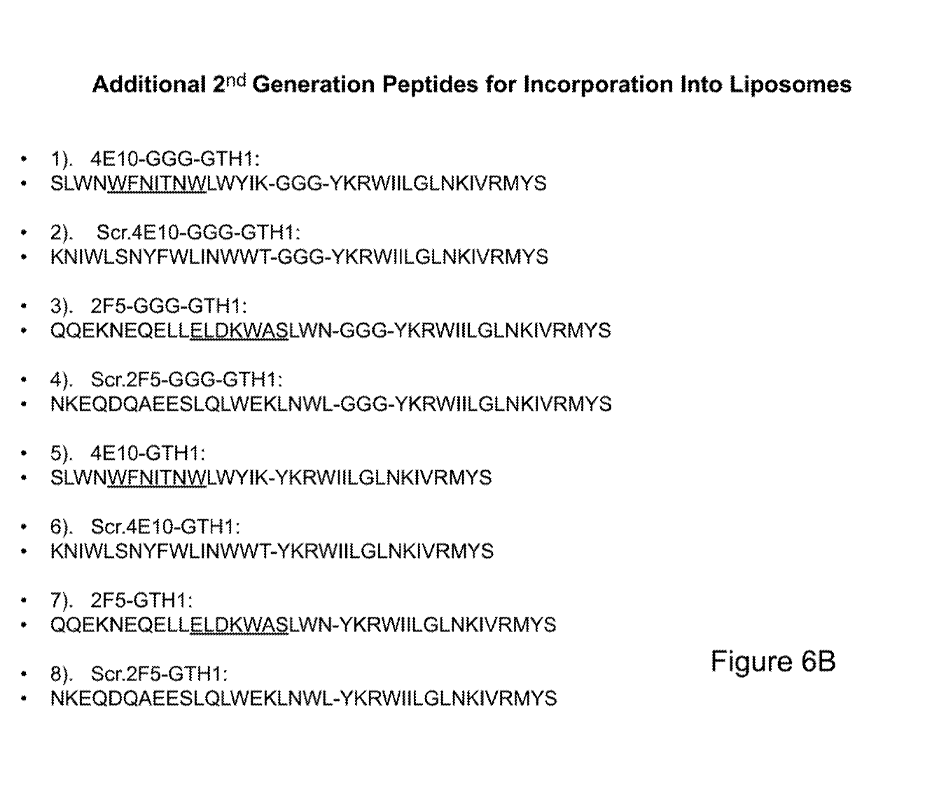

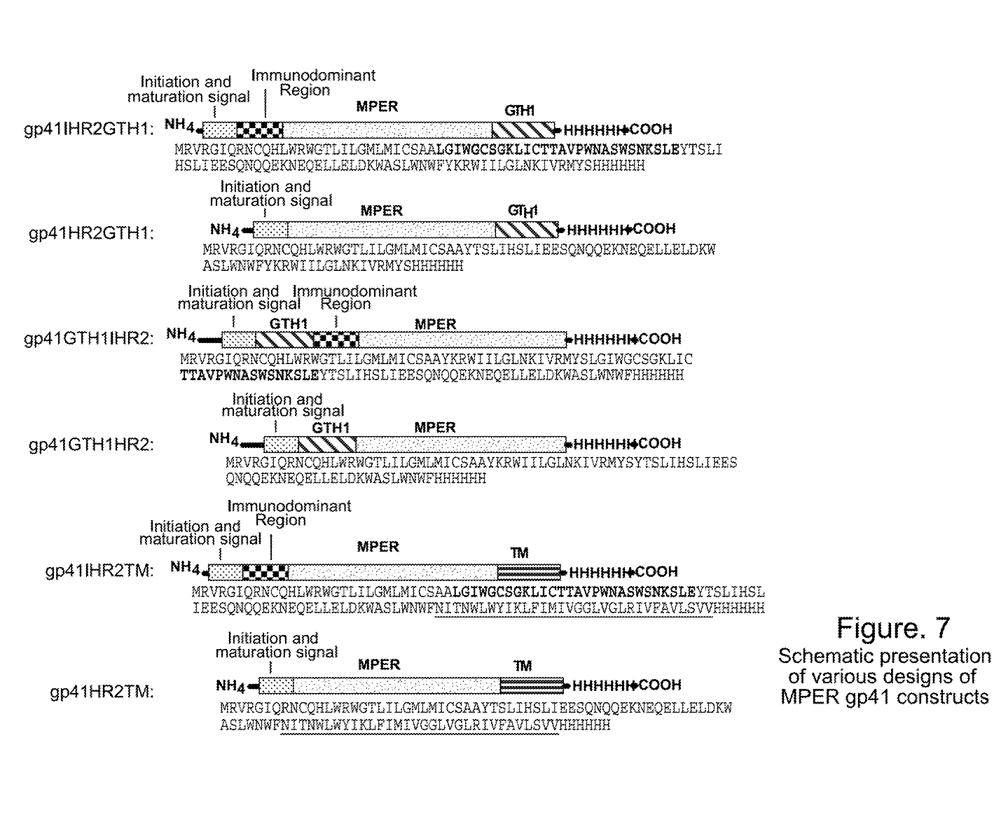

In accordance with this embodiment of the invention, peptide sequences that include a nominal epitope of a broadly neutralizing anti-HIV antibody and a hydrophobic linker, such as GTH1 (see FIG. 6 for sequence), are embedded into synthetic liposomes. In a preferred aspect, the nominal epitope is that of mAbs 2F5 (ELDKWAS) or 4E10 (WFNITNW), which, as noted above, lie in the MPER of HIV-1 envelope gp41. The epitope can be present in the peptide such that antibodies specific therefor have relatively unconstrained access or, alternatively, the epitope can be present in the peptide in relation to the hydrophobic linker so as to mimic the native orientation of the MPER region. Specific examples of peptide sequences suitable for use in the invention are set forth in FIG. 6. In addition, the MPER gp41 region can be expressed as recombinant proteins in recombinant vaccinia virus, in human cell expression systems, and formulated with amphipathic alpha helices at the N or C termini of the gp41 component for ease in association with liposomes (FIG. 7).

Liposomes suitable for use in the invention include, but are not limited to, those comprising POPC, POPE, DMPA (or sphingomyelin (SM)), lysophosphorylcholine, phosphatidylserine, and cholesterol (Ch). While optimum ratios can be determined by one skilled in the art, examples include POPC:POPE (or POPS):SM:Ch or POPC:POPE (or POPS):DMPA:Ch at ratios of 45:25:20:10. Alternative formulations of liposomes that can be used include DMPC (1,2-dimyristoyl-sn-glycero-3-phosphocholine) (or lysophosphorylcholine), cholesterol (Ch) and DMPG (1,2-dimyristoyl-sn-glycero-3-phoshpho-rac-(1-glycerol) formulated at a molar ratio of 9:7.5:1 (Wassef et al, ImmunoMethods 4:217-222 (1994); Alving et al, G. Gregoriadis (ed.), Liposome technology 2^{nd} ed., vol. III CRC Press, Inc., Boca Raton, Fla. (1993); Richards et al, Infect. Immun. 66(6):285902865 (1998)). The above-described lipid compositions can be complexed with lipid A and used as an immunogen to induce antibody responses against phospholipids (Schuster et al, J. Immunol. 122:900-905 (1979)). A preferred formulation comprises POPC:POPS:Ch at ratios of 60:30:10 complexed with lipid A according to Schuster et al, J. Immunol. 122:900-905(1979). Peptides suitable for inclusion in such a formulation include, but are not limited to, 2F5-GTH1, 4E10-GTH1, SP8926-GTH1, and SP8928-GTH1.

The optimum ratio of peptide to total lipid can vary, for example, with the peptide and the liposome. For the peptides of Example 3, a ratio 1:420 was advantageous.

The above-described liposomes can be admixed with recombinant domain V of P2 glycoprotein 1 to elicit antibodies against this domain.

The liposome-peptide conjugates can be prepared using standard techniques (see too Examples 3 and 4 that follow).

The peptide-liposome immunogens of the invention can be formulated with, and/or administered with, adjuvants such as lipid A, oCpGs, TRL4 agonists or TLR 7 agonists that facilitate robust antibody responses (Rao et al, Immunobiol. Cell Biol. 82(5):523 (2004)). Other adjuvants that can be used include alum and Q521 (which do not break existing B cell tolerance). Preferred formulations comprise an adjuvant that is designed to break forms of B cell tolerance, such as oCpGs in an oil emulsion such as Emulsigen (an oil in water emulsion) (Tran et al, Clin. Immunol. 109(3):278-287 (2003)). Additional suitable adjuvants include those described in Ser. No. 11/302,505, filed Dec. 14, 2005, including the TRL agonists disclosed therein.

The peptide-liposome immunogens can be administered, for example, IV, intranasally, subcutaneously, intraperitoneally, intravaginally, or intrarectally. The route of administration can vary, for example, with the patient, the conjugate and/or the effect sought, likewise the dosing regimen. The peptide-liposome immunogens are preferred for use prophylactically, however, their administration to infected individuals may reduce viral load.

As described in Example 3 that follows, the peptide-liposome conjugates can be used as reagents for the detection of MPER-specific B cell responses. For example, the peptide-liposome constructs can be conjugated with a detectable label, e.g., a fluorescent label, such as fluorescein. The fluorescein-conjugated liposomes can be used in flow cytometric assays as a reagent for the detection of anti-MPER specific B cell responses in hosts immunized with HIV-1 Env proteins that present exposed MPER region. These reagents can be used to study peripheral blood B cells to determine the effectiveness of immunization for anti-MPER antibody induction by measuring the number of circulating memory B cells after immunization. The data presented in the Examples that follow indicate that conformational change associated binding of HIV-1 cluster II monoclonal antibodies to nominal epitope peptide lipid conjugates correlates with HIV-1 neutralization (see Example 5).

It will be appreciated from a reading of the foregoing that if HIV has evolved to escape the host immune response by making the immune system blind to it, other infectious agents may have evolved similarly. That is, this may represent a general mechanism of escape. That being the case, approaches comparable to those described herein can be expected to be useful in the treatment of such other agents well.

Certain aspects of the invention are described in greater detail in the non-limiting Examples that follow (see also Maksyutov et al, J. Clin. Virol. December; 31 Suppl 1:S26-38 (2004), US Appin. 20040161429, and Haynes et al, Science 308:1906 (2005)).

This application is related to U.S. application Ser. No. 11/812,992, filed Jun. 22, 2007, U.S. application Ser. No. 11/785,077, filed Apr. 13, 2007, PCT/US2006/013684, filed Apr. 12, 2006, U.S. Prov. Appin. No. 60/670,243, filed Apr. 12, 2005, U.S. Prov. Appin. No. 60/675,091, filed Apr. 27, 2005, U.S. Prov. Appin. No. 60/697,997, filed Jul. 12, 2005, and U.S. Prov. Appin. No. 60/757,478, filed Jan. 10, 2006, the entire contents of which applications are incorporated herein by reference.

### Example 1

Design of an HIV-1 immunogen that can induce broadly reactive neutralizing antibodies is a major goal of HIV-1 vaccine development. While rare human mabs exist that broadly neutralize HIV-1, HIV-1 envelope immunogens do not induce these antibody specificities. In this study, it was demonstrated that the two most broadly reactive HIV-1 envelope gp41 human mabs, 2F5 and 4E10, are polyspecific, autoantibodies reactive with cardiolipin. Thus, current HIV-1 vaccines may not induce antibodies against membrane proximal gp41 epitopes because of gp41 membrane proximal epitopes mimicry of autoantigens.

### Experimental Details

Monoclonal Antibodies.

Mabs 2F5, 2G12, and 4E10 were produced as described (Steigler et al, AID Res. Human Retroviruses 17:1757 (2001), Purtscher et al, AIDS 10:587 (1996), Trkola et al, J. Virol. 70:1100 (1996)). IgG1b12 (Burton et al, Science 266:1024-1027 (1994)) was the generous gift of Dennis Burton, Scripps Institute, La Jolla, Calif. Mab 447-52D (Zolla-Pazner et al, AIDS Res. Human Retrovirol. 20:1254 (2004)) was obtained from the AIDS Reagent Repository, NIAID, NIH. The remainder of the mabs in Table 1 were produced from HIV-1 infected subjects and used as described (Robinson et al, AIDS Res. Human Retrovirol. 6:567 (1990), Binley et al, J. Virol. 78:13232 (2004)).

Autoantibody Assays.

An anti-cardiolipin ELISA was used as described (DeRoe et al, J. Obstet. Gynecol. Neonatal Nurs. 5:207 (1985), Harris et al, Clin. Exp. Immunol. 68:215 (1987)). A similar ELISA was adapted for assay for mab reactivity to phosphatidylserine, phosphatidylcholine, phosphatidyethanolamine, and sphingomyelin (all purchased from Sigma, St. Louis, Mo.). The Luminex AtheNA Multi-Lyte ANA Test (Wampole Laboratories, Princeton, N.J.) was used for mab reactivity to SS-A/Ro, SS-B/La, Sm, ribonucleoprotein (RNP), Scl-70, Jo-1, double stranded (ds) DNA, centromere B, and histone. Mab concentrations assayed were 150 μg, 50 μg, 15 μg, and 5 μg/ml. Ten μl of each concentration (0.15 μg, 0.05 μg, 0.015 μg, and 0.005 μg, respectively, per assay) were incubated with the Luminex fluorescence beads and the test performed per manufacturer's specifications. Values in Table 1 are results of assays with 0.15 μg added per test. In addition, an ELISA for SS-A/Ro (ImmunoVision, Springdale, Ark.) and dsDNA (Inova Diagnostics, San Diego, Calif.) was also used to confirm these autoantigen specificities. Reactivity to human epithelial Hep-2 cells was determined using indirect immunofluoresence on Hep-2 slides using Evans Blue as a counterstain and FITC-conjugated goat anti-human IgG (Zeus Scientific, Raritan N.J.). Slides were photographed on a Nikon Optiphot fluorescence microscope. Rheumatoid factor was performed by nephelometry (Dade Behring, Inc (Newark, Del.). Lupus anticoagulant assay was performed by activated partial thromboplastin (aPTT) and dilute Russell viper venom testing, as described (Moll and Ortel, Ann. Int. Med. 127:177 (1997)). Fourty μl of 1 mg/ml of 2F5, 4E10 and control mabs were added to pooled normal plasma (final mab concentration, 200 μg/ml) for lupus anticoagulant assay. Anti-62 glycoprotein-1 assay was an ELISA (Inova Diagnostics, Inc.). Serum antibodies to dsDNA, SS-A/Ro, SS-B/La, Sm, RNP and histone occur in patients with SLE; serum antibodies to centromere B and scl-70 (topoisomerase I) are found in systemic sclerosis; and antibodies to Jo-1 are found in association with polymyositis (Rose and MacKay, The Autoimmune Diseases, Third Ed. Academic Press, San Diego, Calif. (1998)).

### Results

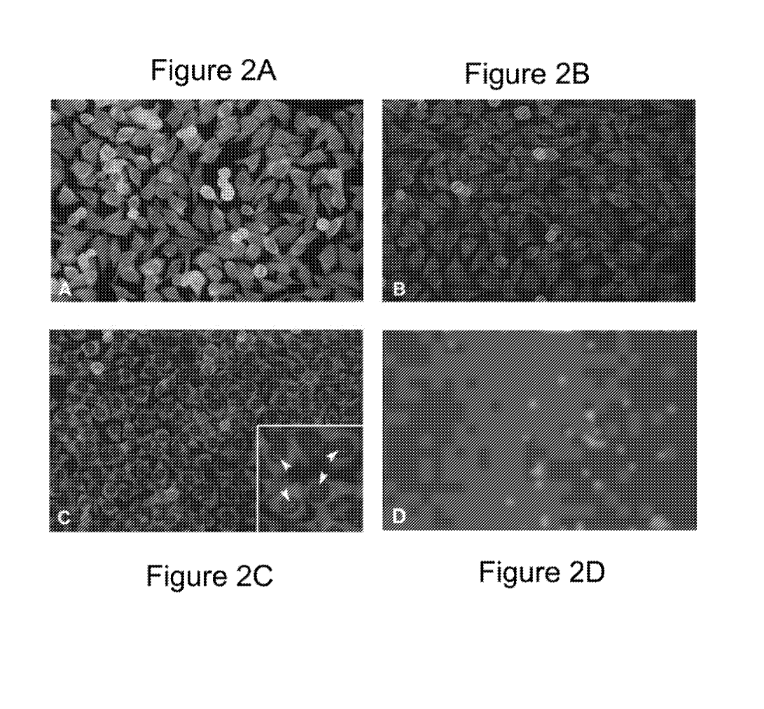

The reactivity of mabs 2F5 and 4E10, two additional rare broadly reactive neutralizing mabs (2012 and IgG1b12), and thirty-one common anti-HIV-1 Env human mabs, with cardiolipin (Robinson et al, AIDS Res. Human Retrovirol. 6:567 (1990)) was determined (Table 1). Both 2F5 and 4E10 reacted with cardiolipin, whereas all 33 of the other mabs were negative. Mab 2F5 also reacted with SS-A/Ro, histones and centromere B autoantigen, while mab 4E10 reacted with the systemic lupus erythematosus (SLE) autoantigen, SS-A/Ro. Both 2F5 and 4E10 reacted with Hep-2 human epithelial cells in a diffuse cytoplasmic and nuclear pattern (Robinson et al, AIDS Res. Human Retrovirol. 6:567 (1990)) (FIG. 2). Thus, both 2F5 and 4E10 are characterized by polyspecific autoreactivity.

TABLE 1

| Mab Type and | | Hep-2 Cell | | | | |
|---|---|---|---|---|---|---|
| Antibody Name | Cardiolipin | Reactivity | Ro(SSA) | dsDNA | Centromere B | Histones |
| Membrane Proximal | 47 | +Cytoplasmic | 290 | — | 1,776 | 1,011 |
| External Region (2F5) | | nuclear | | | | |
| Membrane Proximal | 15,434 | +Cytoplasmic | 221 | — | — | — |
| External Region (4E10) | | nuclear | | | | |
| CD4 Binding | — | +Cytoplasmic | — | 513 | 479 | 185 |
| Site (IgG1b12) | | nucleolar | | | | |
| CD4 Binding Site | — | — | — | — | — | — |
| (F1.5E, 25G) | | | | | | |
| Adjacent CD4 Binding | — | — | — | — | 1,131 | — |
| Site (A32) | | | | | | |
| Adjacent CD4 Binding | — | — | — | 768 | 1,422 | 539 |
| Site (1.4 G) | | | | | | |
| Adjacent CD4 Binding Site | — | — | — | — | — | — |
| (1.4C, 4.6H, 4.11C) | | | | | | |
| Third variable loop (CO11, | — | — | — | — | — | — |
| F2A3, F3.9F, LA21, 447-52D) | | | | | | |
| gp41 immunodominant | — | — | — | — | — | — |
| region (7B2, KU32) | | | | | | |
| gp41 immunodominant | — | +intermediate | — | — | 314 | — |
| region (2.2B) | | filament | | | | |
| C1-C4 gp120 | — | — | — | — | — | — |
| (8.2A, 2.3B) | | | | | | |
| C1-C4 gp120 | — | — | — | — | — | — |
| (EH21, C11) | | | | | | |
| Glycan-dependent | — | — | — | — | — | — |
| (2G12) | | | | | | |
| CCR5 binding site (1.7B, 2.1C, | — | — | — | — | — | — |
| LF17, E51 1.9F, LA15, 4.8E, | | | | | | |
| LA28, 1.9E, E047, 2.5E, ED10) | | | | | | |
| Positive control | 34 | +homogeneous | 1365 | 228 | 624 | 34 |
| serum | | nuclear | | | | |
| Negative controls | <16 | — | <120 | <120 | <120 | <120 |

All Mabs were negative in assays for reactivity with La (SSB), Sm, Scl-70 and Jo-1, except for Ku32 mab that reacted with Sm. Ro (SSA), dsDNA, centromere B, histone and cardiolipin antibody values are in relative units based on a standard curve.
— = negative

Of the two other rare neutralizing mabs, one mab, 2G12, was not autoreactive, while another mab against the CD4 binding site, IgG1 b12 (Stiegler et al, AIDS Res. Hum. Retroviruses 17:1757 (2001)), reacted with ribonucleoprotein, dsDNA, and centromere B as well as with Hep-2 cells in a cytoplamic and nucleolar pattern (Table 1 and FIG. 2). Of the 31 more common anti-HIV-1 mabs studied, only two mabs with specificity for binding near the CD4 binding site (A32, 1.4G) and a mab to a non-neutralizing gp41 epitope (2.2 B) showed evidence of polyreactivity (Table 1).

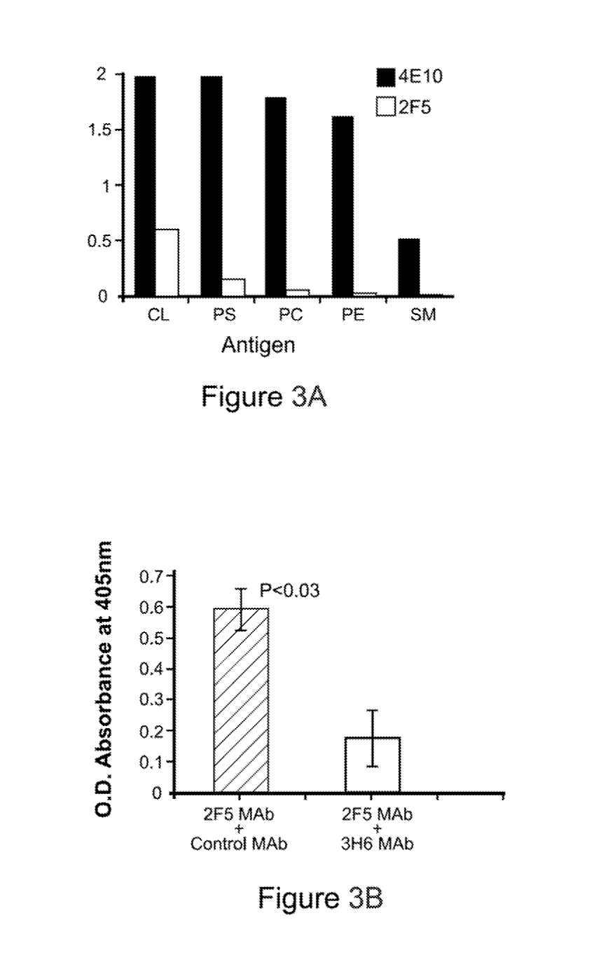

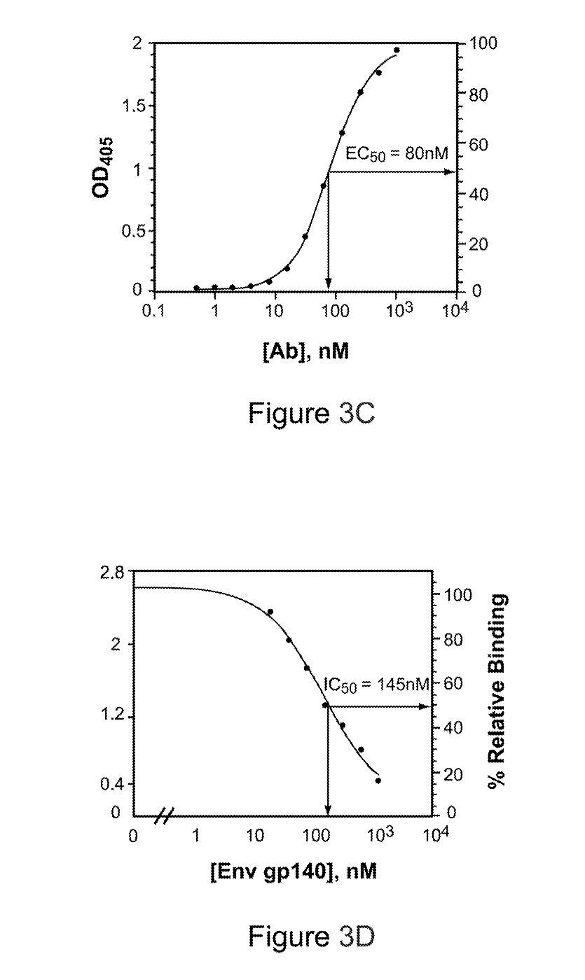

To determine if 2F5 and 4E10 were similar to prothrombotic anti-cardiolipin antibodies found in SLE-associated anti-phospholipid syndrome (Burton et al, Science 266:1024-1027 (1994)), both mabs were tested for lupus anticoagulant activity, and for the ability to bind to prothombin (PT), beta-2 glycoprotein-1, phosphatidylserine (PS), phosphatidylcholine (PC), phosphatidylethanolamine (PE), and sphingomyelin (SM) (Robinson et al, AIDS Res. Human Retrovirol. 6:567 (1990)). Whereas 2F5 was negative for these reactivities, 4E10 had lupus anticoagulant reactivity, and reacted strongly with PS, PC, PE, weakly with SM and PT, and negatively with 132 glycoprotein-1. (See FIG. 3.)

Anti-cardiolipin antibodies can be found in patients with disordered immunoregulation due to autoimmune disease or infection (Burton et al, Science 266:1024-1027 (1994)). Anti-cardiolipin autoantibodies are induced by syphilis, leprosy, leishmaniasis, Epstein Barr virus, and HIV-1 (Burton et al, Science 266:1024-1027 (1994)). Unlike anti-cardiolipin antibodies found in SLE, “infectious” anti-cardiolipin antibodies are rarely prothrombotic, and are transient. Thus, 4E10 is similar to anti-cardiolipin antibodies in autoimmune disease, and 2F5 is similar to anti-cardiolipin antibodies in infectious diseases.

Autoreactive B cell clones with long CDR3 lengths are normally deleted or made tolerant to self antigens ((Zolla-Pazner et al, AIDS Res. Human Retrovirol. 20:1254 (2004)). Thus, HIV-1 may have evolved to escape membrane proximal antibody responses by having conserved neutralizing epitopes as mimics of autoantibody epitopes. These data suggest that current HIV-1 vaccines do not routinely induce robust membrane proximal anti-envelope neutralizing antibodies because antibodies targeting these epitopes are derived from autoreactive B cell clones that are normally deleted or made tolerant upon antigenic stimulation by HIV-1 Env. These observations may also explain the rare occurrence of HIV-1 in SLE patients who may be unable to delete such clones (Fox et al, Arth. Rhum. 40:1168 (1997)).

### Example 2

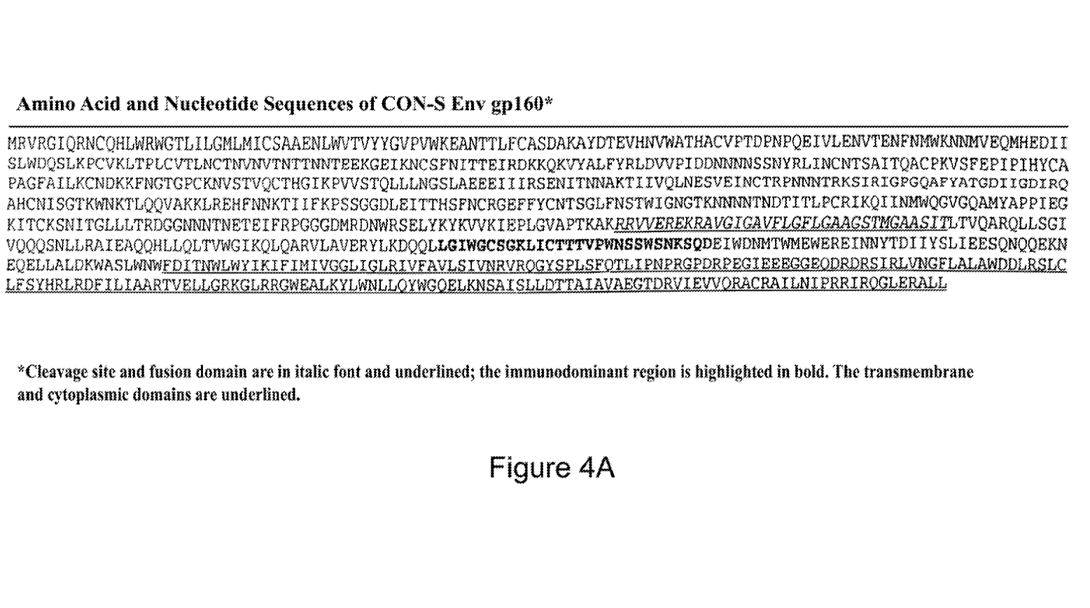

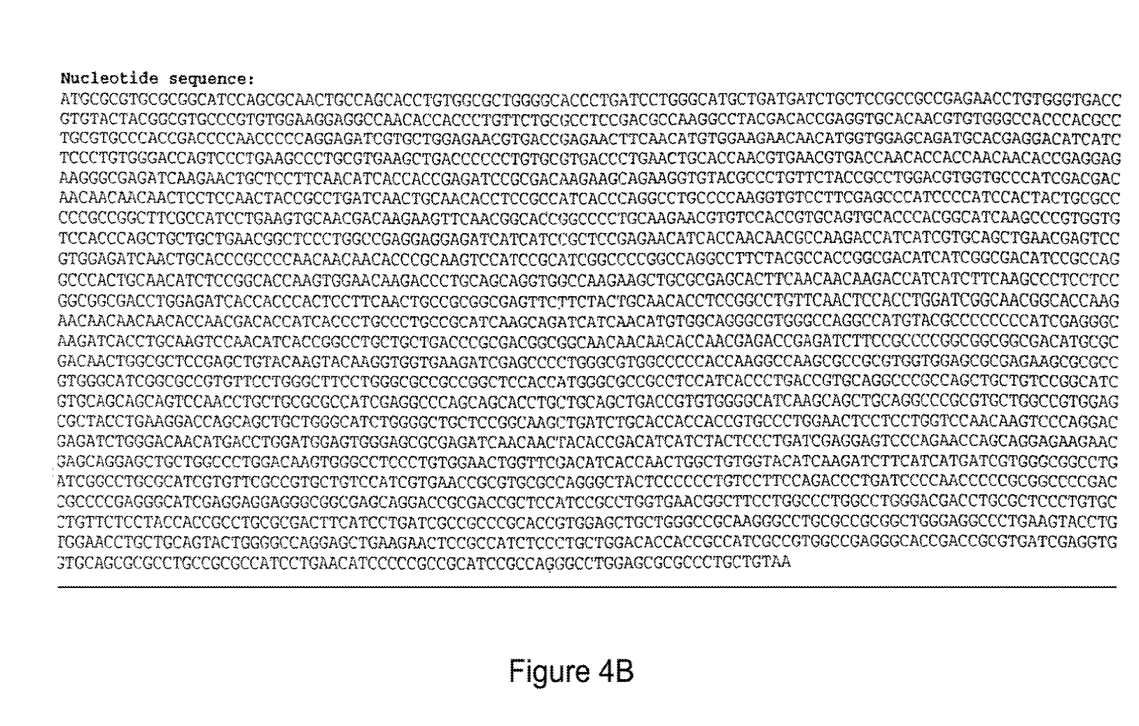

The ability of autoantigens of the invention to induce the production of neutralizing antibodies was studied using, as autoantigen, cardiolipin (lamellar and hexagonal phases), 1-palmitoyl-2-oleoyl-sn-glycero-3-[phospho-L-serine] (POPS) (lamellar and hexagonal phases), 1-palmitoyl-2-oleoyl-phosphatidylethanolamine (POPE) (lamellar phase) and dioleoyl phosphatidylethanolamine (DOPE) (hexagonal phase). Guinea pigs (4 per group) were immunized with phospholopid (cardiolipin lamellar phase, cardiolipin hexagonal phase, POPS lamellar phase, POPS hexagonal phase, POPE lamellar phase or DOPE hexagonal phase) in 10 μg of oCpGs, four times, with each immunization being two weeks apart. Following the four phospholipid immunizations, a final immunization was made IP with 10 μg of oCpGs with 100 μg of group M consensus Env, CON-S gp140CFI oligomer (that is, the CFI form of the protein shown in FIG. 4A).

Neutralization assays were performed using an Env pseudotype neutralization assay in TMZ cells (Wei et al, Nature 422:307-312 (2003), Derdeyn et al, J Virol 74:8358-8367 (2000), Wei et al, Antimicrob Agents Chemother 46:1896-1905 (2002), Platt et al, J Virol 72:2855-2864 (1998), Mascola et al, J. Virol. 79:10103-10107 (2005)), as described below:

### Cell Culture

TZM-bl is an adherent cell line and is maintained in T-75 culture flasks. Complete growth medium (GM) consists of D-MEM supplemented with 10% fetal bovine serum (FBS, heat-inactivated) and gentamicin (50 μg/ml). Cell monolayers are disrupted and removed by treatment with trypsin/EDTA:

### Trypsin-EDTA Treatment for Disruption of TZM-bl Cell Monolayers:

Cell monolayers maintained in T-75 culture flasks are disrupted and removed by treatment with trypsin/EDTA at confluency when splitting cells for routine maintenance and when preparing cells for assay.

1. Decant the culture medium and remove residual serum by rinsing monolayers with 6 ml of sterile PBS.
2. Slowly add 2.5 ml of an 0.25% Trypin-EDTA solution to cover the cell monolayer. Incubate at room temp for 30-45 seconds. Decant the trypsin solution and incubate at 37° C. for 4 minutes. Do not agitate the cells by hitting or shaking the flask while waiting for the cells to detach.
3. Add 10 ml of GM and suspend the cells by gentle pipet action. Count cells.
4. Seed new T-75 culture flasks with approximately 10⁶ cells in 15 ml of GM. Cultures are incubated at 37° C. in a 5% CO₂/95% air environment. Cells should be split approximately every 3 days.

### Virus Stocks

Stocks of uncloned viruses may be produced in either PBMC or T cell lines. Pseudoviruses may be produced by transfection in an appropriate cell type, such as 293T cells. All virus stocks should be made cell free by low speed centrifugation and filtration (0.45-micron) and stored at −80° C. in GM containing 20% FBS.

### TCID50 Determination

It is necessary to determine the TCID50 of each virus stock in a single-cycle infection assay (2-day incubation) in TZM-bl cells prior to performing neutralization assays. A cut-off value of 2.5-times background RLU is used when quantifying positive infection in TCID50 assays.

Too much virus in the neutralization assay can result in strong virus-induced cytopathic effects that interfere with accurate measurements. Most virus stocks must be diluted at least 10-fold to avoid cell-killing. A standard inoculum of 200 TCID50 was chosen for the neutralization assay to minimize virus-induced cytopathic effects while maintaining an ability to measure a 2-log reduction in virus infectivity. It should be noted that different strains vary significantly in their cytopathicity. Virus-induced cytopathic effects may be monitored by visual inspection of syncytium formation under light microscopy. Cytopthic effects may also be observed as reductions in luminescence at high virus doses in the TCID50 assay.

### Neutralizing Antibody Assay Protocol

NOTE 1: All incubations are performed in a humidified 37° C., 5% CO₂ incubator unless otherwise specified.
NOTE 2: Assays with replication-competent viruses are performed in DEAE-GM containing 1 μM indinavir.
1. Using the format of a 96-well flat-bottom culture plate, place 150 μl of GM in all wells of column 1 (cell control). Place 100 μl in all wells of columns 2-11 (column 2 will be the virus control). Place an additional 40 μl in all wells of columns 3-12, row H (to receive test samples).
2. Add 11 μl of test sample to each well in columns 3 & 4, row H. Add 11 μl of a second test sample to each well in columns 5 & 6, row H. Add 11 μl of a third test sample to each well in columns 7 & 8, row H. Add 11 μl of a fourth test sample to each well in columns 9 & 10, row H. Add 11 μl of a fifth test sample to each well in columns 11 & 12, row H. Mix the samples in row H and transfer 50 μl to row G. Repeat the transfer and dilution of samples through row A (these are serial 3-fold dilutions). After final transfer and mixing is complete, discard 50 μl from the wells in columns 3-12, row A into a waste container of disinfectant.
3. Thaw the required number of vials of virus by placing in an ambient temperature water bath. When completely thawed, dilute the virus in GM to achieve a concentration of 4,000 TCID₅₀/ml.

Cell-free stocks of virus should be prepared in advance and cryopreserved in working aliquots of approximately 1 ml,

4. Dispense 50 μl of cell-free virus (200 TCID₅₀) to all wells in columns 2-12, rows A through H. Mix by pipet action after each transfer. Rinse pipet tips in a reagent reservoir containing 40 ml sterile PBS between each transfer to avoid carry-over.
5. Cover plates and incubate for 1 hour.
6. Prepare a suspension of TZM-bl cells (trypsinize approximately 10-15 minutes prior to use) at a density of 1×10⁵ cells/ml in GM containing DEAE dextran (37.5 μg/ml). Dispense 100 μl of cell suspension (10,000 cells per well) to each well in columns 1-12, rows A though H. Rinse pipet tips in a reagent reservoir filled with sterile PBS between each transfer to avoid carry-over. The final concentration of DEAE dextran is 15 μg/ml.
7. Cover plates and incubate for 48 hours.
8. Remove 150 μl of culture medium from each well, leaving approximately 100 μl. Dispense 100 μl of Bright Glom′ Reagent to each well. Incubate at room temperature for 2 minutes to allow complete cell lysis. Mix by pipet action (at least two strokes) and transfer 150 μl to a corresponding 96-well black plate. Read the plate immediately in a luminometer.
9. Percent neutralization is determined by calculating the difference in average RLU between test wells (cells+serum sample+virus) and cell control wells (cells only, column 1), dividing this result by the difference in average RLU between virus control (cell+virus, column 2) and cell control wells (column 1), subtracting from 1 and multiplying by 100. Neutralizing antibody titers are expressed as the reciprocal of the serum dilution required to reduce RLU by 50%.

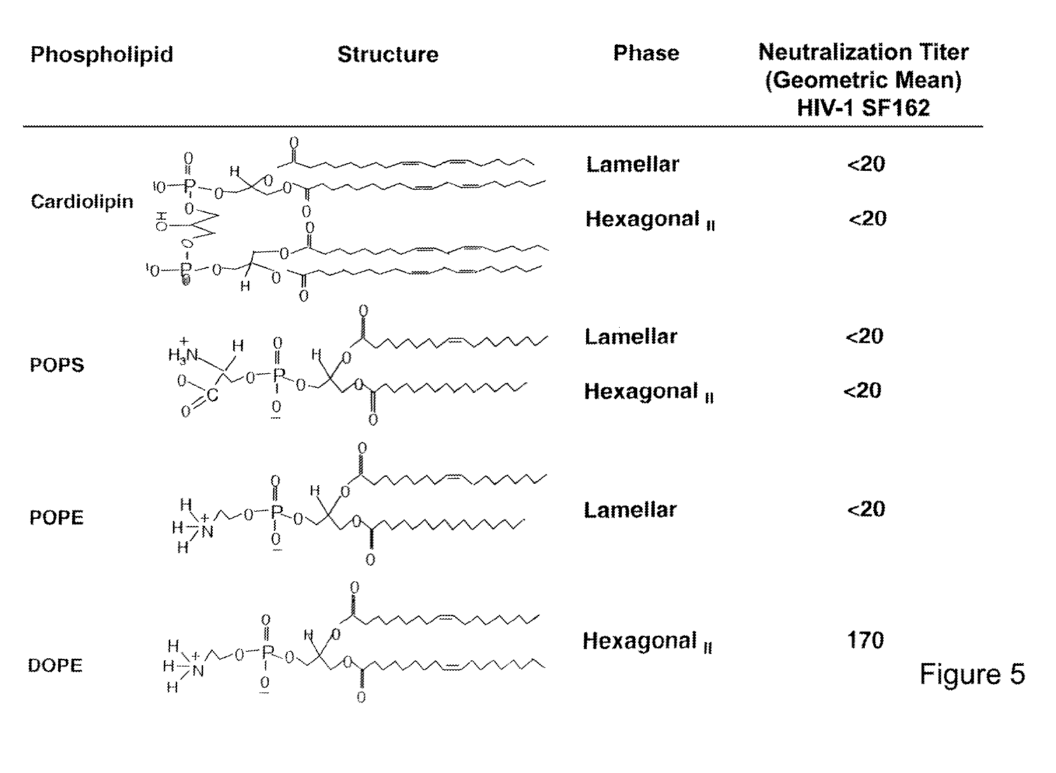

As shown in FIG. 5, animals receiving DOPE (hexagonal phase) had a neutralization titer of 170.

### Example 3

### Immunogen Design

Peptide sequences that include the nominal epitopes of mAbs 2F5 and 4E10, respectively, linked to a hydrophobic linker (GTH1) were synthesized and embedded into synthetic liposomes (FIG. 6). The first generation of immunogens was designed with the 2F5 and 4E10 epitope sequences at the distal end of the lipid bilayer (FIG. 6A). These constructs provided unconstrained access of mAbs to their respective epitopes. The second generation constructs have been designed to mimic the native orientation of the MPER region with the 2F5 and 4E10 mAb epitope sequences linked proximal to the hydrophobic linker (FIGS. 6A, 6B).

The composition of the synthetic liposomes comprised the following phospholipids, POPC (1-Palmitoyl-2-Oleoyl-sn-Glycero-3-Phosphocholine), POPE (1-Palmitoyl-2-Oleoyl-sn-Glycero-3-Phosphoethanolamine), DMPA (1,2-Dimyristoyl-sn-Glycero-3-Phosphate), and Cholesterol dissolved in chloroform (purchased from Avanti Polar Lipids (Alabaster, Ala.).).

Synthetic liposomes were prepared by dispensing appropriate molar amounts of phospholipids (POPC:POPE:DMPA:Ch=45:25:20:10) in chloroform resistant tubes. The phospholipids were mixed by vortexing and the mixture was dried in the fume hood under a gentle stream of nitrogen. Any residual chloroform was removed by storing the lipids under a high vacuum (15 h). Aqueous suspensions of phospholipids were prepared by adding PBS or TBS buffer, pH 7.4, and incubating at 37° C. for 10-30 minutes, with intermittent, vigorous vortexing to resuspend the phospholipids. The milky, uniform suspension of phospholipids was then sonicated in a bath sonicator (Misonix Sonicator 3000, Misonix Inc., Farmingdale, N.Y.). The sonicator was programmed to run 3 consecutive cycles of 45 seconds of total sonication per cycle. Each cycle included 5 seconds of sonication pulse (70 watts power output) followed by a pulse off period of 12 seconds. At the end of sonication, the suspension of lamellar liposomes was stored at 4° C.

HIV-1 MPER peptides GTH1-2F5 and GTH1-4E10 (FIG. 6) were dissolved in 70% chloroform, 30% methanol. Chloroform solutions of lipids were added to the peptide solution, in the molar ratios of 45:25:20:10 (POPC:POPE:DMPA:Cholesterol). Each peptide was added to a ratio of peptide:total phospholipids of 1:420. The mixture was vortexed, then dried and resuspended as described above.

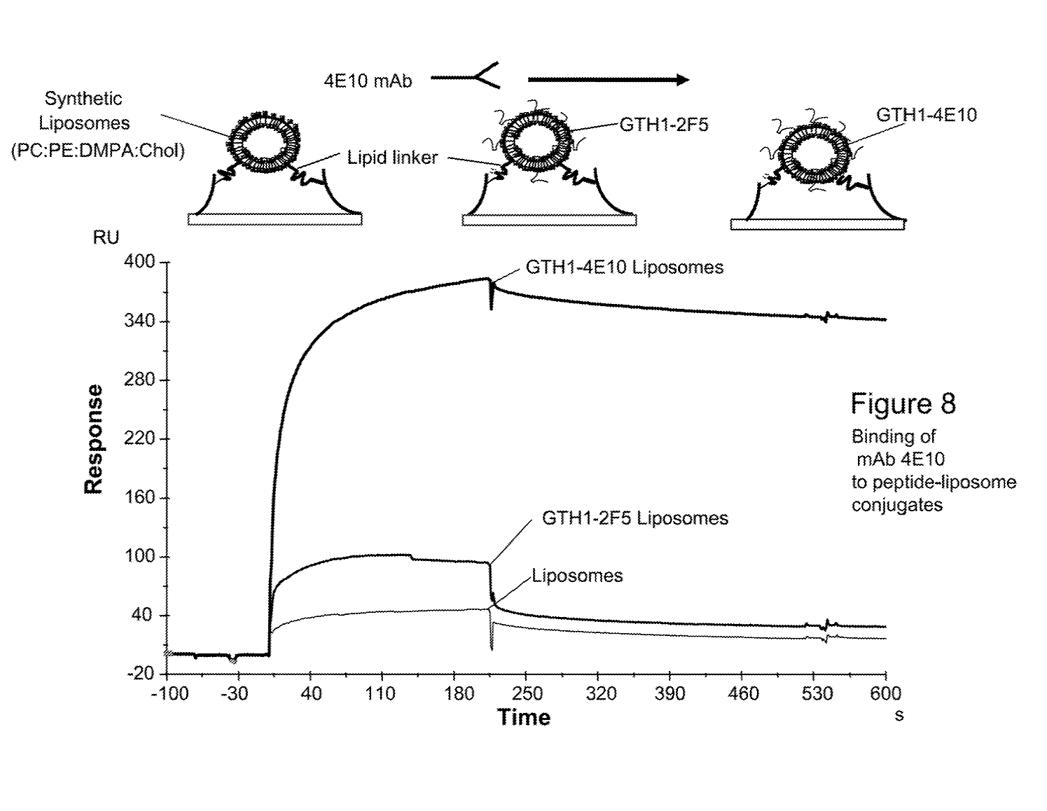

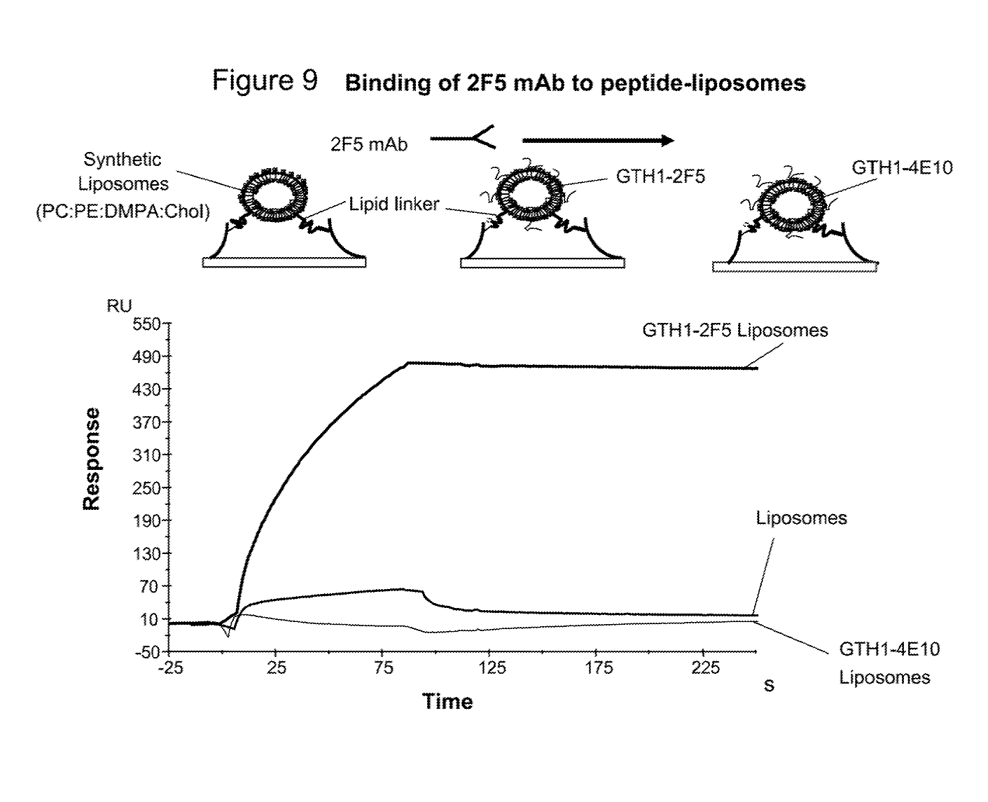

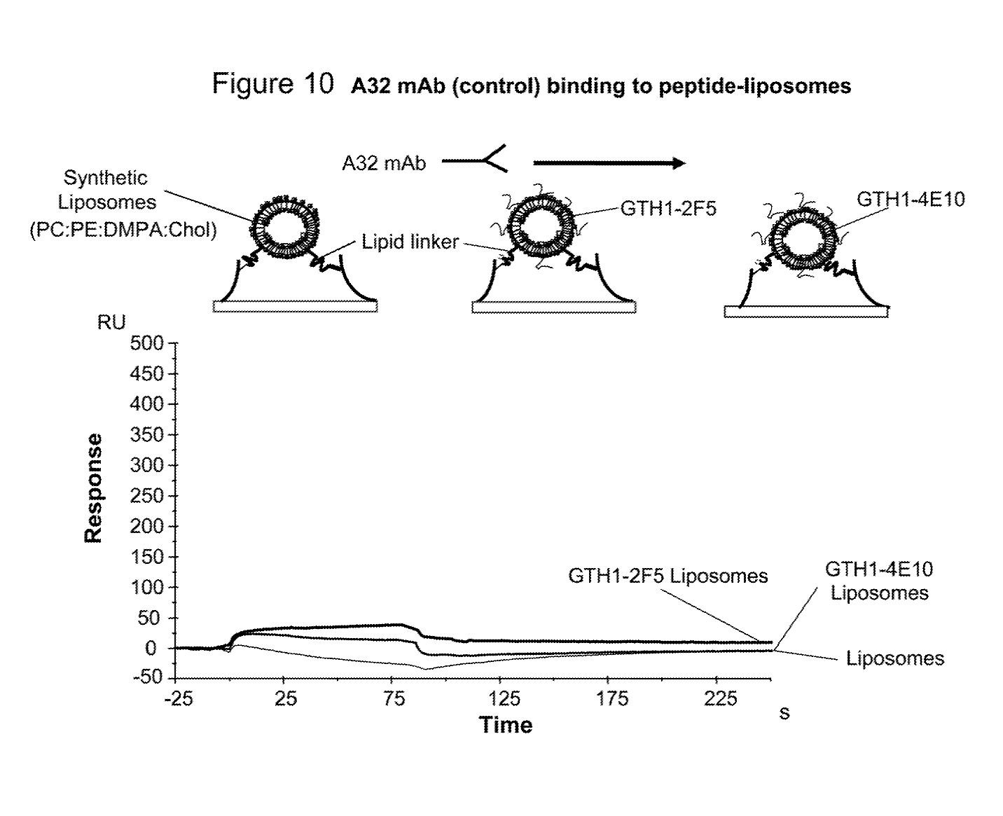

Binding assays to test specificity of mAb binding to each peptide-lipid conjugate were performed following capture of the liposomes on a BAcore L1 sensor chip, which allows immobilization of lipid bilayer via a hydrophobic linker. 2F5, 4E10 and control mAbs (A32 or 17b) were injected over each of the sensor surfaces with either synthetic liposomes, or peptide-lipid conjugates and the binding monitored on a BIAcore 3000 instrument (FIGS. 8-11).

### Immunization Strategy

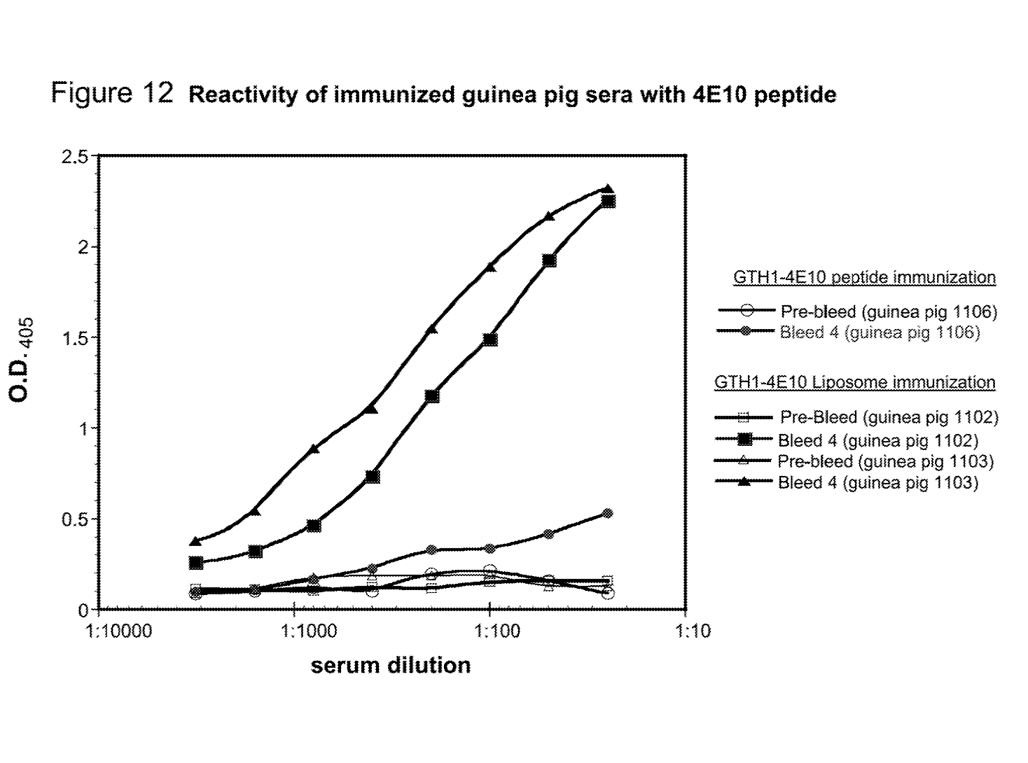

The immunization strategy incorporated a regimen that allows temporary breaks in tolerance. The protocol involves the use of oCpGs, the TLR9 ligand that has been used to break tolerance for the production of anti-dsDNA antibodies in mice (Tran et al, Clin. Immunol. 109(3):278-287 (2003)). The peptide-liposome conjugates were mixed (1:1) with the adjuvant, Emulsigen plus oCpG. The Emulsigen mixed adjuvant (2×) was prepared by mixing 375 μL of Emulsigen, 250 μL of oCpG and 625 μL of saline. Each guinea pig was immunized on a 21-day interval with 250 μg of either peptide alone or peptide-liposome conjugates with equivalent amount of peptide. Serum samples were harvested as pre-bleed prior to first immunization and at each subsequent immunizations. Serum samples were analyzed by ELISA assay (FIG. 12) for binding to peptide epitopes and for viral neutralization assay (Table 2). Data in FIG. 12, show strong reactivity to 4E10 peptide of sera from two guinea pigs immunized with GTH1-4E10 liposomes, while only low level of reactivity was observed in a serum from 4E10 peptide immunized animal. Both the positive sera also neutralized HIV-1 MN strain (Table 2).

TABLE 2
Induction of neutralizing antibodies in guinea pigs immunized
with 4E10 peptide-liposomes

HIV-1 Strain/
antibody titer

| Animal No. | MN | SS1196 |
|---|---|---|
| 1102 Bleed 4 | 209 | 32 |
| 1103 Bleed 4 | 60 | <20 |

### Application of Peptide-Liposome Conjugates in the Detection of Antigen Specific B Cell Responses.

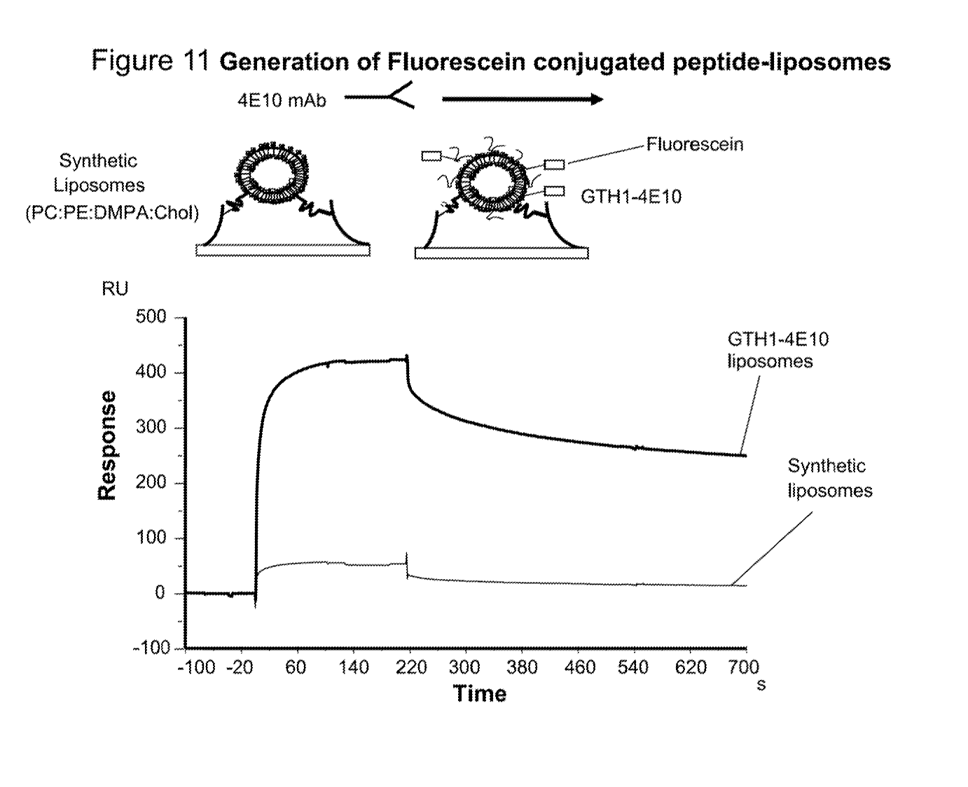

The above peptide-liposome conjugates have been utilized as a reagent for the detection of MPER specific B cell responses. The peptide-liposome constructs (2F5 and 4E10) were conjugated with fluorescein by incorporating fluorescein-POPE in the lipid composition, The flourescein-POPE was mixed with unconjugated POPE at a ratio of 45:55 and then mixed with the rest of the lipids in the molar ratio as described above. In BIAcore binding assays, both fluorescein conjugated 2F5 and 4E10-peptide-liposomes retained their specificity in binding to their respective mAbs (FIG. 11).

### Example 4

### Generation of Peptide-Lipid Conjugates

Phospholipids POPC (1-Palmitoyl-2-Oleoyl-sn-Glycero-3-Phosphatidylcholine), POPE (1-Palmitoyl-2-Oleoyl-sn-Glycero-3-Phosphatidylethanolamine), DOPE (1,2-Dioleoyl-sn-Glycero-3-Phosphatidylethanolamine); DMPA (1,2-Dimyristoyl-sn-Glycero-3-Phosphate) and cholesterol dissolved in chloroform were purchased from Avanti Polar Lipids (Alabaster, Ala.). Phospholipid liposomes were prepared by dispensing appropriate molar amounts of phospholipids in chloroform resistant tubes. Chloroform solutions of lipids were added to the peptide solution, in molar ratios of 45:25:20:10 (POPC:POPE:DMPA:Cholesterol). HIV-1 membrane proximal peptides were dissolved in 70% chloroform, 30% methanol. Each peptide was added to a molar ratio of peptide:total phospholipids of 1:420. The phospholipids were mixed by gentle vortexing and the mixture was dried in the fume hood under a gentle stream of nitrogen. Any residual chloroform was removed by storing the lipids under a high vacuum (15 h). Aqueous suspensions of phospholipids were prepared by adding PBS or TBS buffer, pH 7.4 and kept at a temperature above the Tm for 10-30 minutes, with intermittent, vigorous vortexing to resuspend the phospholipids followed by Sonication in a bath sonicator (Misonix Sonicator 3000, Misonix Inc., Farmingdale, N.Y.). The sonicator was programmed to run 3 consecutive cycles of 45 seconds of total sonication per cycle. Each cycle included 5 seconds of sonication pulse (70 watts power output) followed by a pulse off period of 12 seconds. At the end of sonication, the suspension of lamellar liposomes was stored at 4° C. and was thawed and sonicated again as described above prior to capture on BIAcore sensor chip.

Design of Peptide-Lipid Conjugates.

Peptides were synthesized and purified by reverse-phase HPLC and purity was confirmed by mass spectrometric analysis. Peptides used in this study include the following—HIV-1 gp41 2F5 epitope peptides—2F5-GTH1 (QQEKNEQELLELDKWASLWN-YKRWIILGLNKIVRMYS); and HIV-1 gp41 4E10 epitope peptides—4E10-GTH1 (SLWNWFNITNWLWYIK-YKRWIILGLNKIVRMYS). Additional peptides to be incorporated into liposomes include—SP8926-GTH1 (EQELLELDKWASLWN-YKRWIILGLNKIVRMYS); and Sp8928-GTH1 (KWASLWNWFDITNWL-YKRWIILGLNKIVRMYS).

Peptide-lipid conjugates. Each of these peptides will be incorporated into synthetic liposomes of varying composition which include:

i) POPC:POPE:DMPA:Cholesterol

ii) POPC:POPS

iii) POPC:POPS:lysoPC

iv) POPC:POPE:Sphingomyelin:Cholesterol

The liposomes will be complexed with and without monophosphoryl Lipid A (Avanti Polar Lipids).

### Example 5

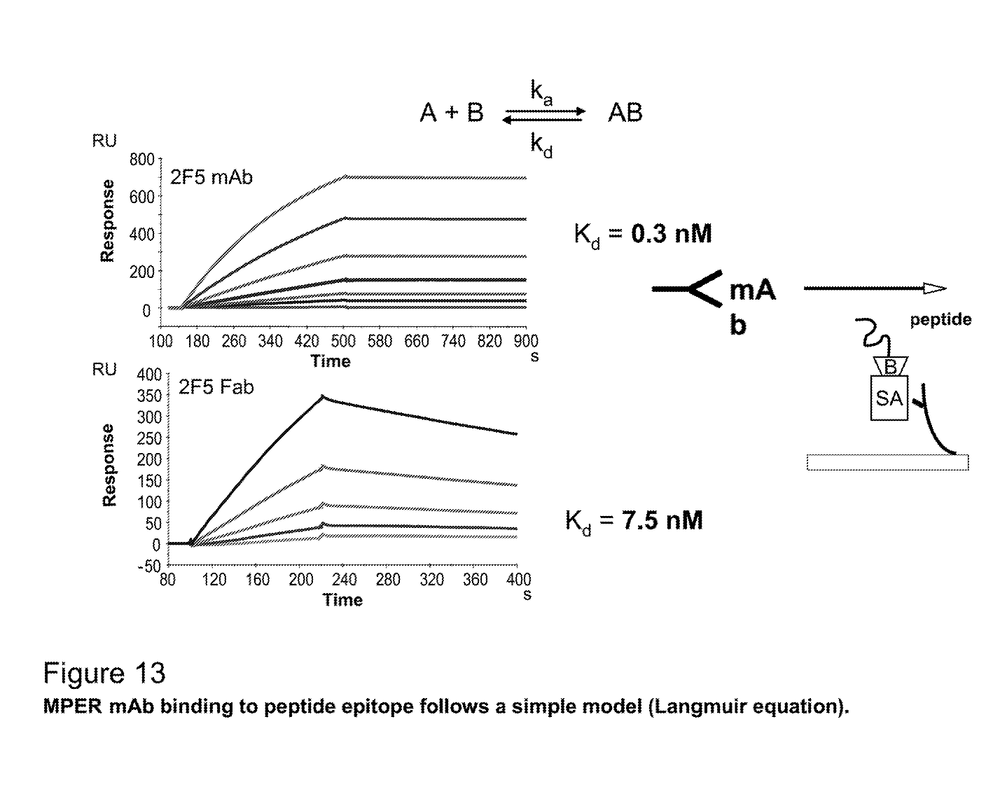

Biotinylated 2F5 nominal epitope peptide (SP62) was anchored on streptavidin coated BIAcore sensor chip (SA) and either 2F5 mab or 2F5 Fab was injected over the peptide surfaces. Specific binding of 2F5 mAb (46.6-1800 nM) or 2F5 Fab (120-2000 nM) was derived following subtraction of non-specific signal on a HR-1 peptide control surface. Kd was calculated following global curve fitting to a simple Langmuir equation using the BlAevaluation software. The data presented in FIG. 13 show that MPER mAb binding to peptide epitope follows a simple model (Langmuir equation).

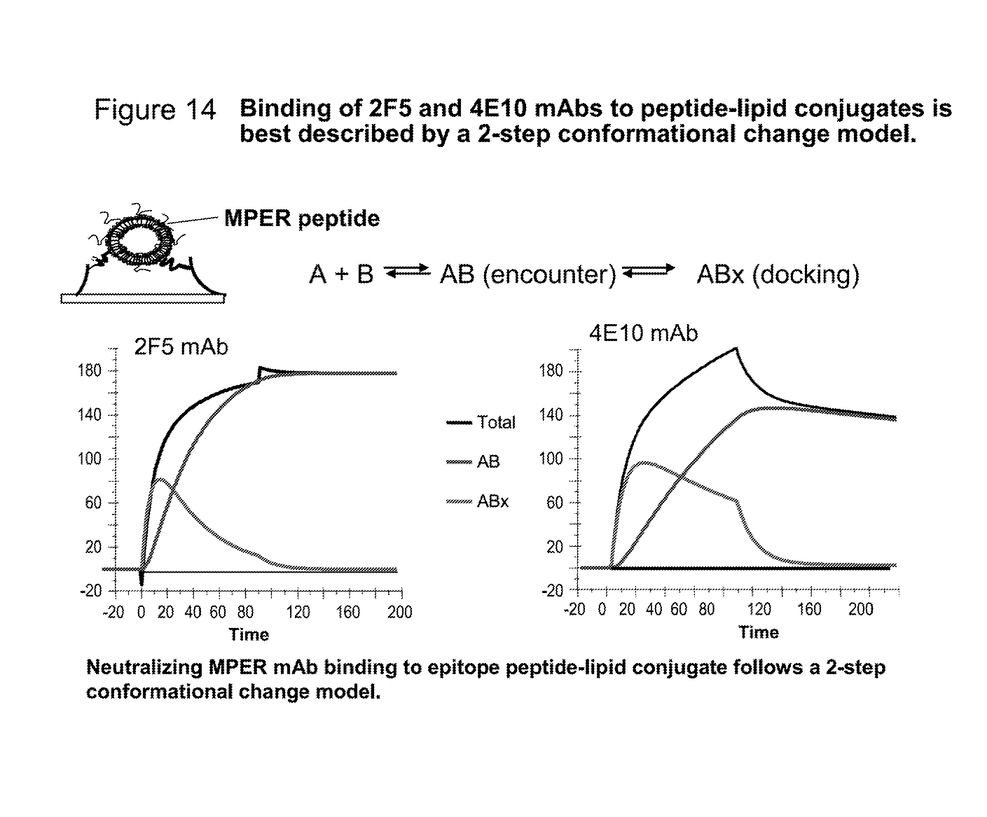

About 600 RU of either 2F5 peptide-lipid (FIG. 14, left panel) or 4E10 peptide-lipid conjugates were anchored to a BIAcore L1 sensor chip and then 2F5 mAb or 4E10 mAb was injected at 100 μg/mL. Curve fitting analysis show that binding of both Mab bound to peptide-lipid conjugates follow a 2-step conformational change mode (FIG. 14). In each of the overlay, the binding data is shown in black and represents the observed total binding response. The component curves for the encounter complex (red) and the docked complex (blue) were simulated from the experimentally determined rate constants.

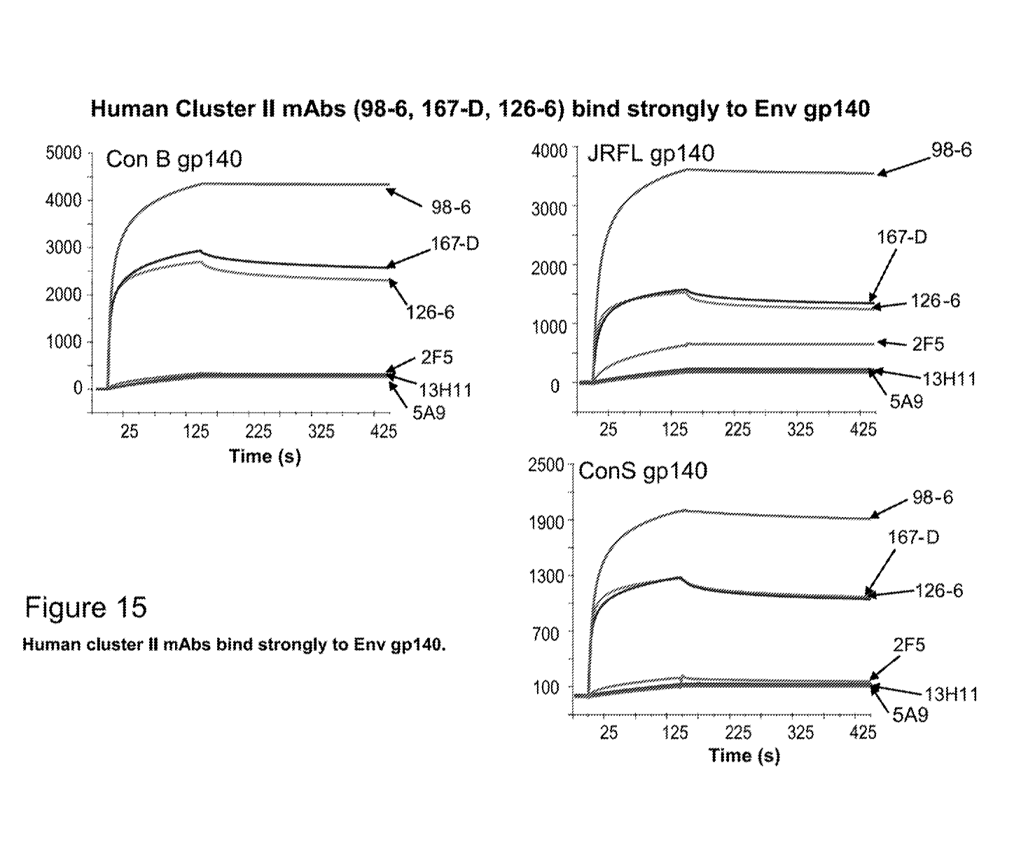

Envelope gp140 oligomers were anchored on a BIAcore CM5 chip and each of the mAbs indicated in FIG. 15 were injected over each of the Env surfaces. Human cluster II mAbs, 98-6, 126-6, and 167-D bound strongly to Env gp140, while no binding was detected with the non-neutralizing murine MPER mAbs, 2F5, and 4E10.

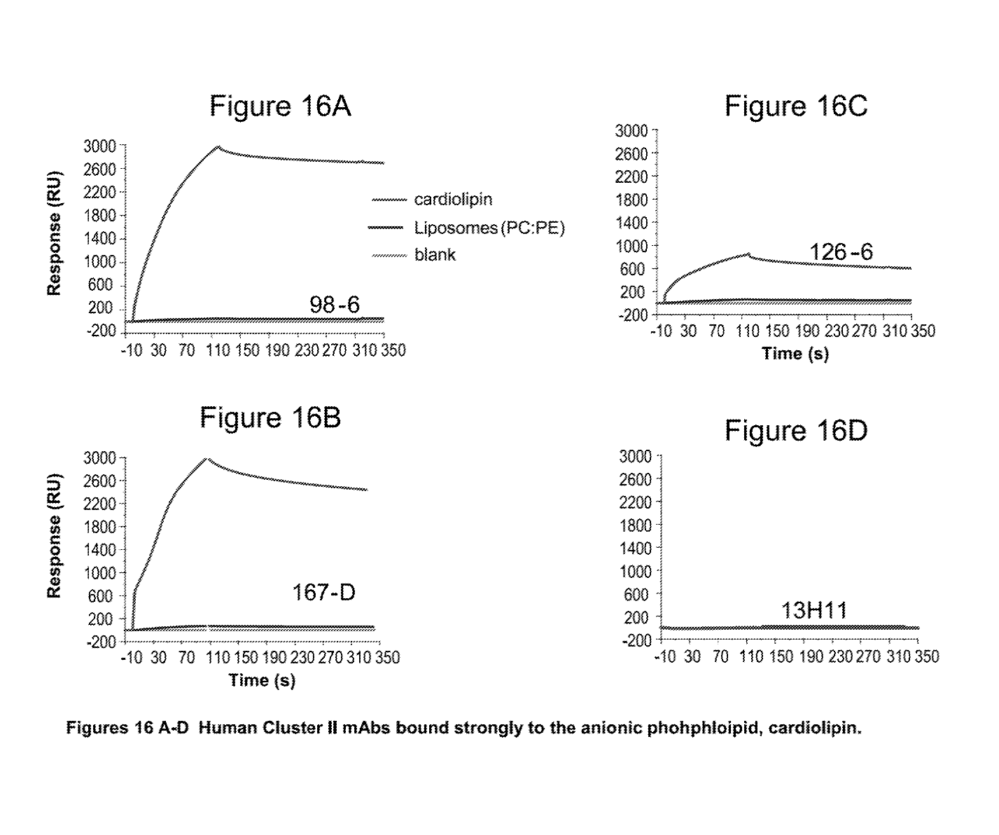

Synthetic liposomes (PC:PE; green), or cardiolipin (red) was anchored on a BIAcore L1 sensor chip through hydrophobic interactions with the lipid linker (FIG. 16). Each of the indicated mAbs (500 nM) was injected over each of the lipid surface and a blank control surface. Strong binding of Cluster II mAb 98-6 and 167-D and moderate binding of mAb 126-6 is shown (FIGS. 16A-C). No binding of the anti-MPER mAb 13H11 to either lipid was observed.

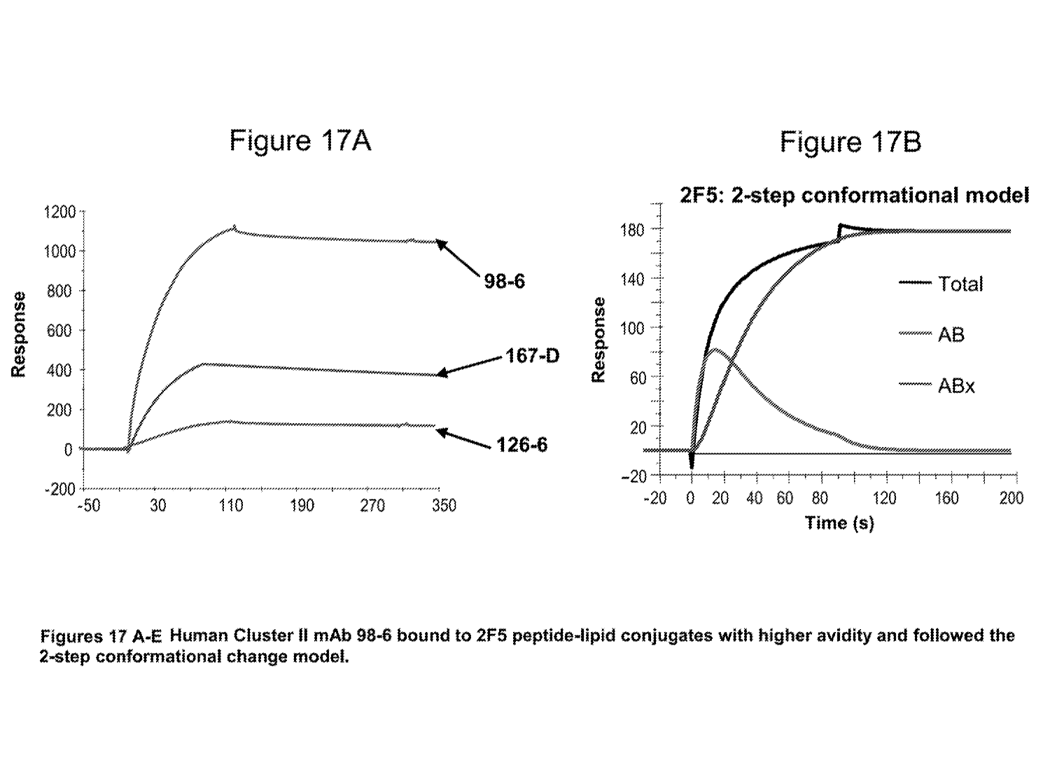

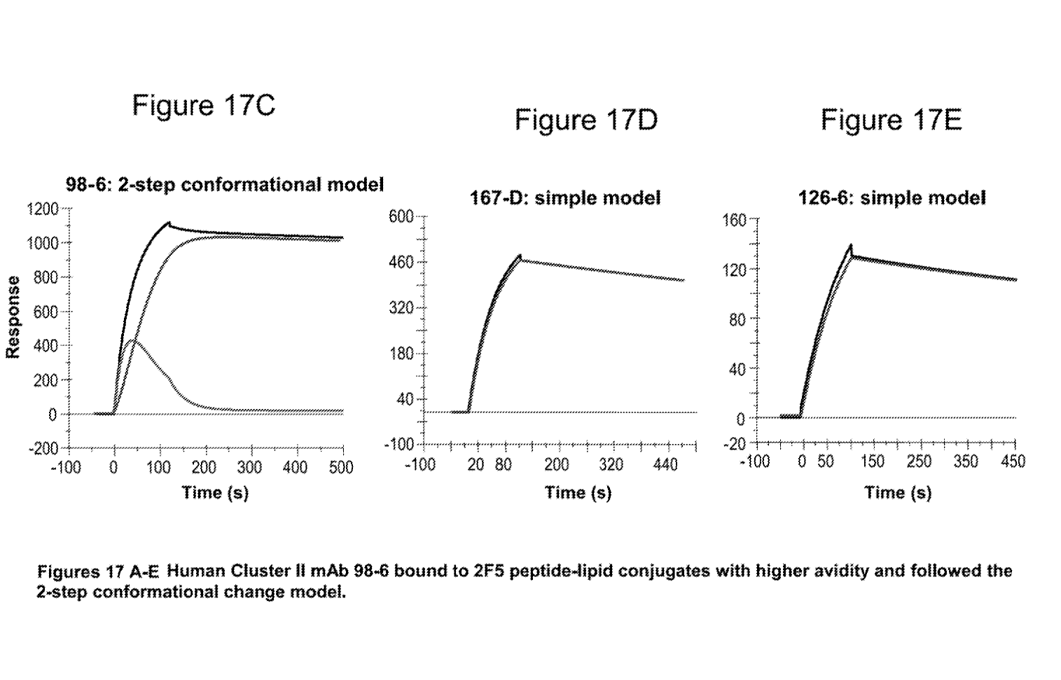

2F5-peptide (SP62) lipid conjugates were anchored to a BIAcore L1 surface and binding to mAb 98-6, 167-D or 126-6 was monitored (FIG. 17A). Mab 98-6 bound strongly to the peptide-lipid conjugates, while relatively lower avidity binding was detected with mAb 167-D and 126-6. Curve fitting analysis show a 2-step conformational change associated binding of 2F5 (FIG. 17B) and 98-6 (FIG. 17C); while the binding of mAbs 167-D (FIG. 17D) and 126-6 (FIG. 17E) followed a simple model (Langmuir equation).

The data presented in Table 3 show binding and neutralization characteristics of 25F and other prototype anti-MPER cluster II mAbs. Only mAb 2F5 and 98-6, which bound strongly to linear epitope peptide and followed a 2-step conformational change model, neutralized HIV-1 in a PBMC assay.

TABLE 3

| | Nominal | | | | HIV |
|---|---|---|---|---|---|
| | Epitope | Env gp140 | Phospholipid | Peptide-Lipid | Neutralization |
| MAb | (HR-2 peptide) | JRFL | Cardiolipin | Conjugates | ID₅₀ In PBMC assay |
| 2F5 | ++ | ++ | + | 2-step | 1 μg/mL |
| | | | | conformational | |
| 98-6 | ++ | ++ | +++ | 2-step | 3.5 μg/mL |
| | | | | conformational | |
| 126-6 | + | ++ | +++ | Simple model | Non-Neut* |
| 167-D | + | ++ | ++ | Simple model | Non-Neut* |
| 13H11 | + | + | −ve | +/− | >50 μg/mL |
| 5A9 | + | + | −ve | +/− | >50 μg/mL |

*Corny et al, J. Virol. 74: 6168 (2000); Nyambi et al, J. Virol. 74: 2096 (2000)

### Example 6

Human monoclonal antibodies (termed CL1, IS4 and IS6) derived from patients with anti-phospholipid syndrome have been studied. (See Table 4.) (Giles et al, J. Immunol. 177: 1729-1736 (2006), Zhu et al, Brit. Jour. Haematol. 105:102-109 (1999), Chukwuocha et al, Mol. Immunol. 39:299-311 (2002), Zhu et al, Brit. Jour. Haematol. 135:214-219 (2006), Pierangeli et al, Thromb. Haemost. 84:388-395 (2000), Lin et al, Arth Rheum 56:1638 (2007), Alam et al, J. Immunol. 178:4424-4435 (2007), Zhao et al, Arth. Rheum. 42:2132-2138 (1999), Lu et al, Arth. Rheum. 52:4018-4027 (2005)). IS4 and IS6 are pathogenic anti-lipid antibodies whereas CL1 is a non-pathogenic anti-lipid autoantibody (Table 4). Whereas none of these antibodies neutralized HIV pseudoviruses in the pseudovirus inhibition assay that reflects primarily infection by virion-cell fusion (Li et al, J. Virol. 79:10108-25 (2005) (Table 5), all three of these antibodies neutralized HIV-1 in the PBMC HIV neutralization assay that depends on endocytosis of HIV and is a mirror of HIV infectivity of CD4 cells in vivo (Table 6). That CL1 neutralized HIV evidences the facts that: a) humans can make non-pathogenic anti-lipid antibodies that neutralize HIV, and b) CL1 is an antibody that can be safely used as a therapeutic Mab for treatment of HIV infected subjects or in the setting of post-exposure prophylaxis of subjects following needle, sexual or other exposure to HIV or HIV infected materials.

TABLE 4
MAbs Derived From an Anti-Phospholipid
Syndrome Patient

| | Antibody Name | | |
|---|---|---|---|
| Antibody Reactivity | CL1 | IS4 | IS6 |
| cardiolipin/PS | ++ | ++ | +/− |
| β-2-glycoprotein-1 domain | 5 | 1 | − |
| prothrombin | − | − | +++ |
| thrombosis in viva in a mouse | − | +++ | ++ |
| model | | | |
| pathogenic MAb | No | Yes | Yes |

TABLE 5
Neutralization of HIV-1 in Pseudovirus Assay
by Anti-Membrane MAbs

| | ID50 in Pseudovirus Assay (μg/mL) |
|---|---|
| MAb | B.6535 |
| Humanized Anti-PS | >50 |
| (Bavituximab) | |
| Control (Erbitux) | >50 |
| Anti-CL (IS4) | >50 |
| Anti-CL/PS (CL1) | >50 |
| Anti-CL/prothrombin (IS6) | >50 |

TABLE 6
Neutralization of HIV-1 Primary Isolates by
Anti Membrane Antibodies

| | IC 80 Neutralization Levels, ug/ml | | | | |
|---|---|---|---|---|---|
| HIV-1 Isolates | CL1 | IS4 | IS6 | Anti-RSV | Tri-Mab* |
| B.Torno | 0.6 | 0.6 | 5 | >50 | 0.03 |
| B.PAVO | 0.3 | 0.3 | 1.6 | >50 | 0.01 |
| B.6535 | 0.06 | 0.06 | 0.62 | ND | ND |
| C.DU123 | 0.4 | 0.6 | 4.6 | >50 | >50 |
| C.DU156 | 2.6 | 2.6 | 11.6 | >50 | >50 |
| C.DU151 | 4.1 | 5.2 | >50 | >50 | >50 |
| C.DU172 | 0.6 | 0.9 | 4.1 | >50 | >50 |
| SHIV SP162P3 | 0.06 | 0.2 | 0.46 | >50 | 0.9 |
| SHIV 89.6P | >50 | 50 | >50 | >50 | 1.8 |
| SIV MAC239 | >50 | >50 | >50 | ND | ND |

*TRI-Mab = 2F5, 2G12, 1b12 Mabs

Alving and colleagues have made a mouse mab against phosphatidyl inositol phosphate and have shown that it neutralizes HIV in a PBMC assay (Wassef et al, Mol. Immunol. 21: 863-868 (1984), Brown et al, Virol. 81: 2087-2091 (2007), Beck et al, Biochem. Biophys Res. Comm. 354: 747-751 (2007)). What the present studies show is that humans can spontaneously make anti-lipid antibodies and that these antibodies can broadly neutralize HIV in an unprecendented manner.

Summarizing, autoimmune disease patients can make antibodies that bind to virus-infected cells and, presumably, to budding HIV virions by virtue of their reactivity to HIV membranes and host membranes. Certain anti-lipid antibodies from autoimmune disease patients can also react with the Envelope trimer (such as IS6) but not all of the antibodies react also with the trimer (i.e., CL1 and IS4 do not react). Therefore, reactivity with the HIV envelope is not a prerequisite for neutralization in these antibodies.

These studies also demonstrate that it may be possible to safely stimulate the production of CL1 like antibodies in humans using gp41 lipid complexes (Alam et al, J. Immunol. 178:4424-4435 (2007), Schuster et al, J. Immunol. 122:900-905 (1984)).

### Example 7

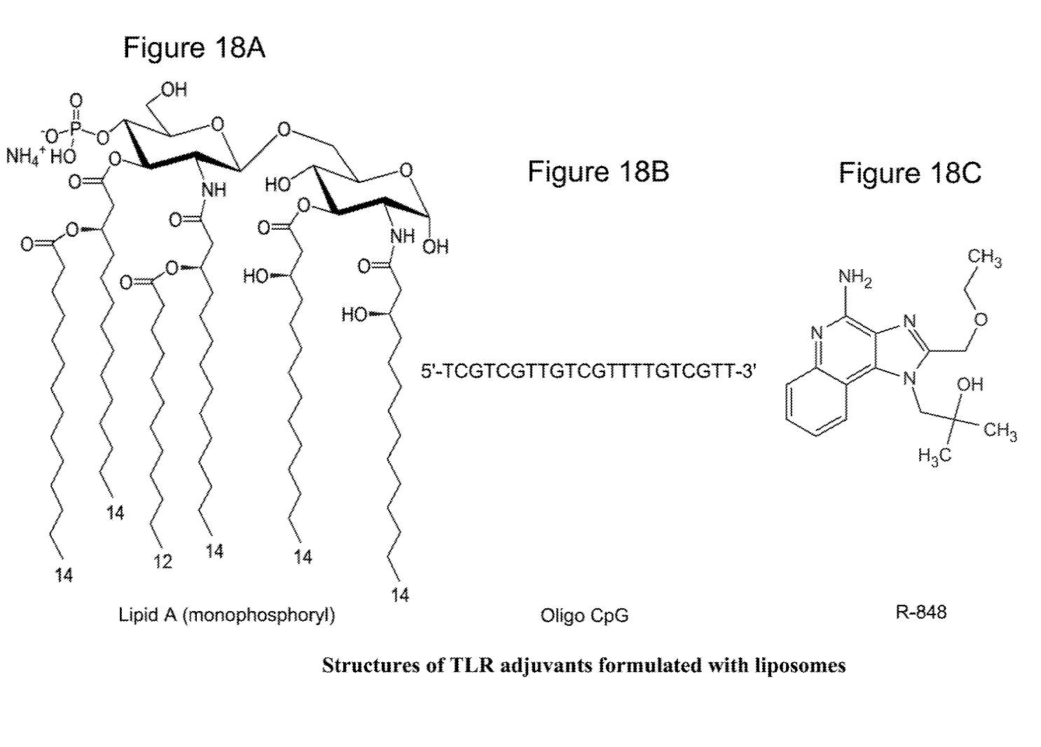

Toll like receptor ligands, shown in FIG. 18, were formulated into liposomal forms with gp41 MPER peptide immunogens.

The construction of Lipid A and R-848 containing MPER peptide liposomes utilized the method of co-solubilization of MPER peptide having a membrane anchoring amino acid sequence and synthetic lipids 1-Palmitoyl-2-Oleoyl-sn-Glycero-3-Phosphocholine (POPC), 1-Palmitoyl-2-Oleoyl-sn-Glycero-3-Phosphoethanolamine (POPE), 1,2-Dimyristoyl-sn-Glycero-3-Phosphate (DMPA) and cholesterol at mole fractions 0.216, 45.00, 25.00, 20.00 and 1.33, respectively. Appropriate amount of MPER peptide dissolved in chloroform-methanol mixture (7:3 v/v), Lipid A dissolved in chloroform or R-848 dissolved in methanol, appropriate amounts of chloroform stocks of phospholipids were dried in a stream of nitrogen followed by over night vacuum drying. Liposomes were made from the dried peptide-lipid film in phosphate buffered saline (pH 7.4) using extrusion technology. Construction of oligo-CpG complexed MPER peptide liposomes used the cationic lipid 1-Palmitoyl-2-Oleoyl-sn-Glycero-3-ethylphospho choline (POEPC) instead of POPC. Conjugation of oCpG was done by mixing of cationic liposomes containing the peptide immunogen with appropriate amounts of oCpG stock solution (1 mg/ml) for the desired dose.

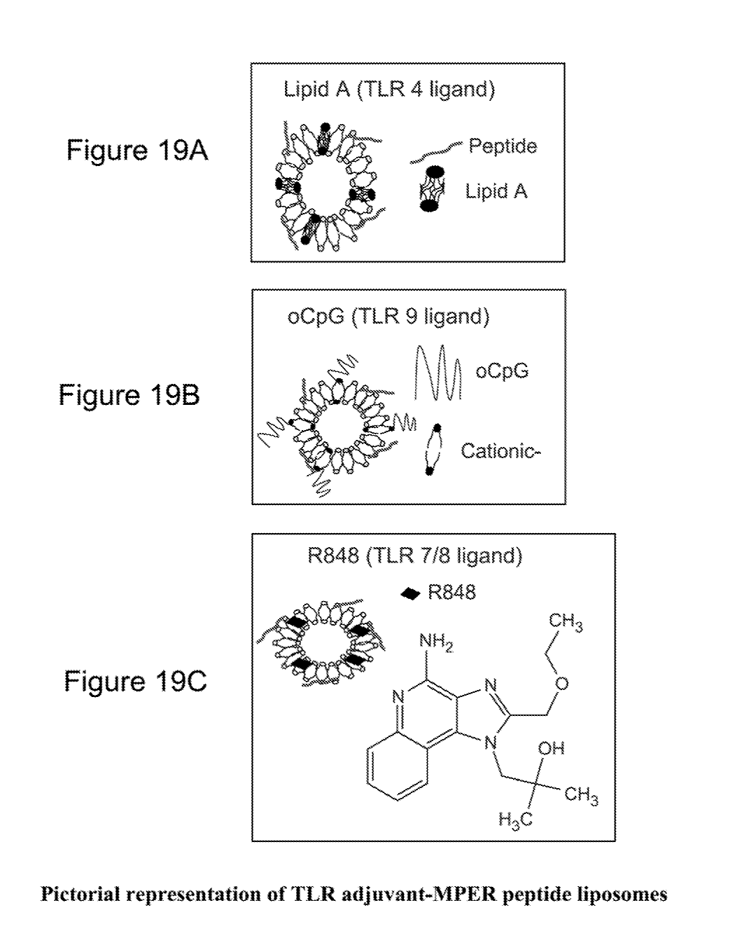

A schematic of the designs displayed in FIG. 19 shows the peptide-liposomes containing different TLR adjuvants; TLR4 (Lipid A); TLR9 (oCpG) and TLR7 (R848).

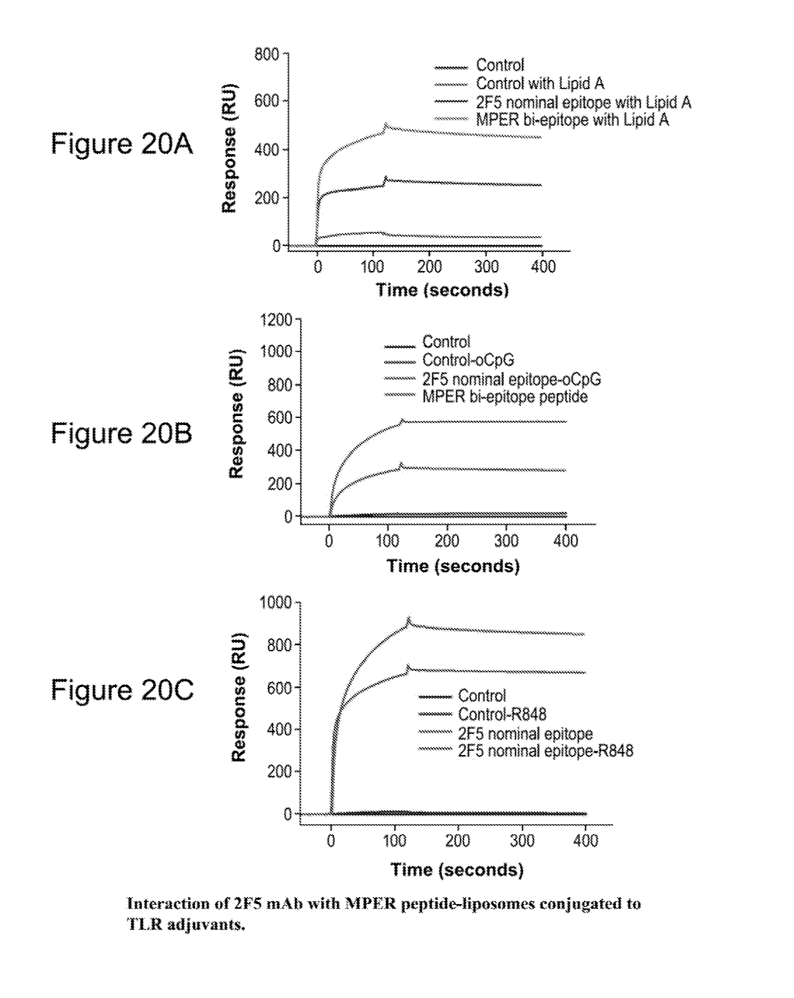

Biacore assay for the binding of 2F5 mAb to its epitope in the peptide-liposome constructs revealed that incorporation or conjugation of TLR adjuvants does not affect binding of HIV neutralizing antibody 2F5. Strong binding of both mAbs 2F5 and 4E10 was observed. (See FIG. 20.)

### Example 8

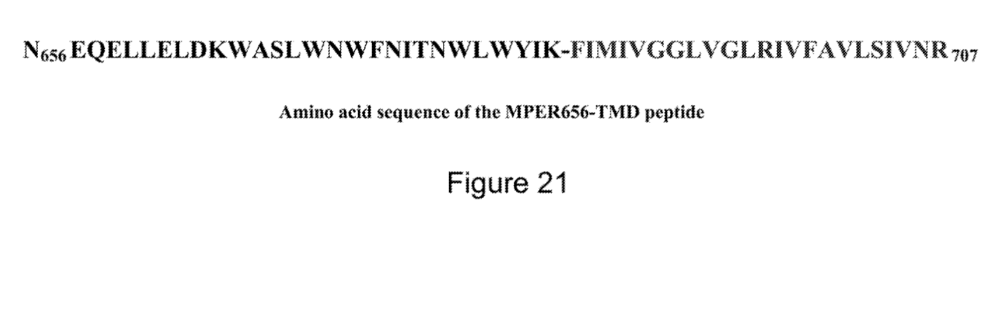

The HIV-1 gp41 membrane proximal external region that precedes the transmembrane domain is the target for the broadly neutralizing antibodies 2F5 and 4E10. The fact that the MPER peptide partitions into membrane interfaces and the lipid reactivity of the antibodies 2F5 and 4E10 led to the design of MPER peptide-liposome conjugates as candidate immunogens for the induction of broadly neutralizing gp41 MPER antibodies. The peptide-liposome conjugation strategy used here involved the design of a synthetic peptide, MPER656-TMD (FIG. 21), corresponding to the MPER that contains the epitopes for both 2F5 and 4E10 mAbs and the incorporation of the transmembrane domain of HIV-1 gp41 (residues 656 to 707 of the gp160).

The MPER656-TMD peptide-liposome conjugate construction involved co-solubilization of MPER656-TMD peptide and synthetic lipids 1-Palmitoyl-2-Oleoyl-sn-Glycero-3-Phosphocholine (POPC), 1-Palmitoyl-2-Oleoyl-sn-Glycero-3-Phosphoethanolamine (POPE), 1,2-Dimyristoyl-sn-Glycero-3-Phosphate (DMPA) and cholesterol at mole fractions 0.43, 45.00, 25.00, 20.00 and 1.33, respectively. An appropriate amount of MPER656-TMD peptide dissolved in chloroform-methanol mixture (8:2 v/v), mixed with appropriate amounts of chloroform stocks of phospholipids was dried in a stream of nitrogen followed by over night vacuum drying. Liposomes were made from the dried peptide-lipid film in phosphate buffered saline (pH 7.4) using extrusion technology.

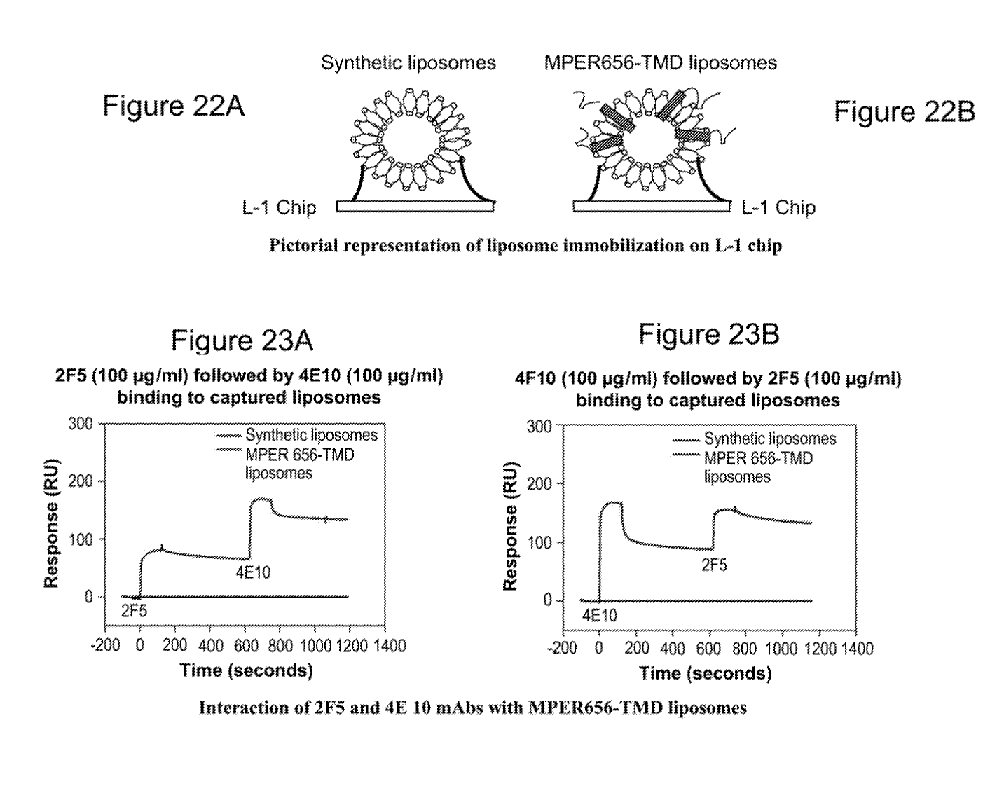

To assess the presentation of MPER epitopes on the TMD liposome constructs, MPER656-GTH1 and peptide free synthetic liposomes were captured on the Biacore L-1 chip that had 3000 RU BSA immobilized on each flow cell (FIG. 22).

Testing of functional presentation of MPER region in the MPER656-TMD-liposome construct involved examining the interaction of 2F5 and 4E10 mAbs with the liposomes immobilized on the Biacore L-1 chip shown in FIG. 23. Peptide specific binding of 2F5 mAb followed by that of 4E10 mAb or vice versa (FIG. 23) confirmed the functional presentation of their respective epitopes contained in the MPER656-TMD peptide.

Thus, 2F5 and 4E10 bound strongly to the gp41 MPER-TMD construct. Therefore, this strategy provides a novel means to present gp41 MPER anchored via the native TMD. The MPER656-TMD peptide that contains the amino acid sequence of the HIV-1 gp41 MPER and transmembrane domain (residues 656 thru 707 of gp160) was used to conjugate the MPER peptide to synthetic liposomes successfully. The functional display of epitopes of both 2F5 and 4E10 mAbs in MPER656-TMD-liposome conjugate makes this construct a very promising immunogen to test for the induction of 2F5 and 4E10 like antibodies.

### Example 9

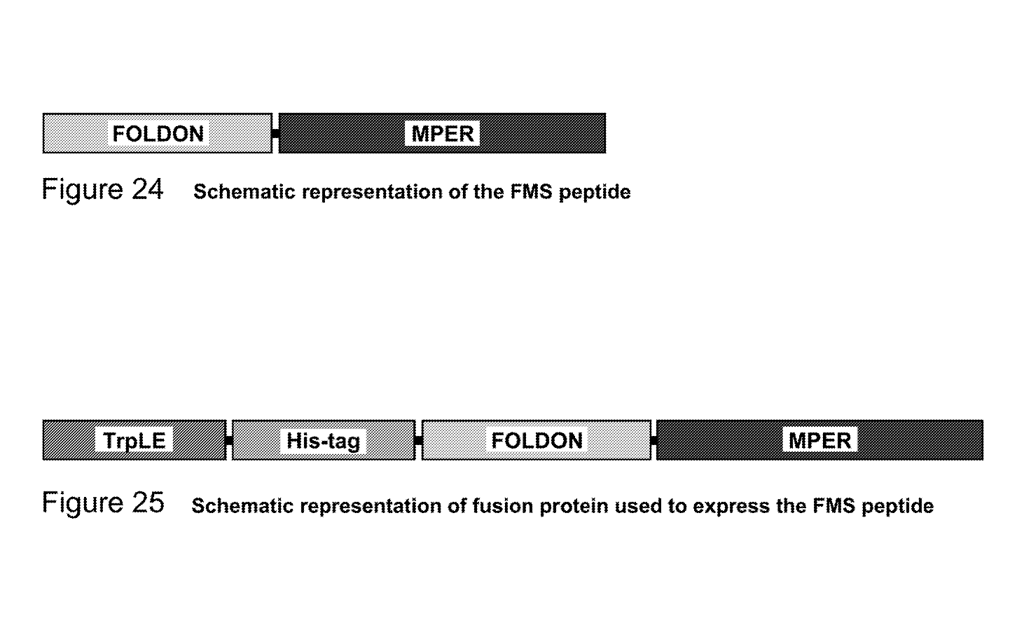

A molecular conjugate has been designed to present the MPER of the HIV-1 coat protein gp41 as a trimer attached to biological membrane mimetics. The peptide (NEQELLELDKWASLWNWFNITNWLWYIK (SEQ ID NO:29)) includes the epitopes for the broadly neutralizing antibodies 2F5, Z13, and 4E10 (the peptide sequence selected can conform to other clades of HIV). The construct is trimerized with an N-terminal foldon domain from T4 fibritin (Papanikolopoulou et al, Methods Mal. Biol. 474:15-33 (2008)), allowing the MPER to adopt a conformation similar to the pre-fusion intermediate state of the virus that is believed to be the target for neutralizing antibodies. The trimer binds to both detergent micelles and phospholipid bilayer liposomes directly using the native peptide sequence at the C-terminus of the MPER. This represents the closest representation to date of the natural presentation of the possible gp41 MPER intermediate state on the HIV-1 membrane. The MPER peptide construct is designated as the FMS peptide below. A schematic representation of the FMS peptide is shown in FIG. 24.

### Synthesis and Preparation

The FMS construct can be expressed and purified from E. coli as a fusion with a TrpLE domain and a 6-Histidine tag, as represented in FIG. 25. The DNA sequence encoding the FMS peptide was ordered from IDT with restriction sites for nde1 and xho1. The plasmid pTCLE is a T7 expression vector that contains a modified TrpLE fusion peptide and a 6 histidine-tag (Yansura, Methods Enzymol. 185:161-166 (1990), Calderone et al, J. Mol. Biol. 262:407-412 (1996)). The DNA encoding the FMS peptide was inserted into pTCLE using the nde1 site that immediately follows the histidine tag and the xho1 site located in the multiple cloning site. This produced a plasmid that contained FMS as a fusion to the TrpLE peptide with an intervening methionine.

The plasmid was transformed into C41(DE3) E. coli cells. The cells were grown to an OD₆₀₀ ˜0.5, when fusion protein expression was induced by the addition of 1 mM IPTG. The cells were allowed to grow for an additional 4 hours, after which the cells were harvested by centrifugation. For a 1 liter growth, the cell pellets were lysed by incubating in 20 mL BUGBUSTER (Pierce) reagent with 100 μg/mL lysozyme and 200 μg/mL DNase for 30 minutes. Cell clumps were broken up by sonication using a Misonix 3000 sonicator equipped with a microtip. After lysis, the inclusion bodies were separated from the soluble protein by centrifugation at 15,000×g for 30 minutes. The inclusion bodies were washed with 10 mL of BUGBUSTER reagent, then centrifuged at 15,000×g for 30 minutes. The washed inclusion bodies were dissolved in wash buffer containing 6M guanidine-HCl in 50 mM sodium phosphate, 10 mM imidazole pH 8.0. The solubilized inclusion bodies were centrifuged at 15,000×g for 30 minutes to remove any debris that is not soluble in 6M guanidine.

The TrpLE fusion protein was purified from other insoluble proteins present in the inclusion bodies with a 5 mL Ni-SEPHAROSE column (GE Healthcare). The TrpLE fusion protein was bound to the column in wash buffer and eluted from the column using elution buffer that contained 250 mM imidazole, 6M guanidine-HCl, and 50 mM sodium phosphate pH 8.0. Following elution, fractions were pooled, and β-mercaptoethanol was added to a final concentration of 1% v/v.

The purified fusion protein was dialyzed against ddH₂O until the protein precipitated, ˜2 hrs. The insoluble protein was pelleted by centrifugation at 15,000×g for 20 minutes, and the supernatant removed. The protein pellet was washed with ddH₂O and dried under vacuum. The protein was dissolved in 70% trifluoroacetic acid at a concentration of between 10 and 20 mg/mL. The cleavage reaction was initiated by the addition of solid cyanogen bromide to a final concentration of 1 M, and incubated for 2 hours at 25° C. The cleavage reaction was stopped by drying the sample under vacuum until all liquid was removed. The cleaved FMS peptide was purified from the TrpLE leader sequence and unreacted fusion protein by capturing the TrpLE and unreacted protein on a Ni-SEPHAROSE column under similar denaturing conditions as used above.

To produce FMS containing micelles, guanidine was removed from the purified FMS peptide using dialysis against ddH₂O overnight. Following dialysis, sodium phosphate buffer at pH 7.0 was added to a final concentration of 50 mM. The precipitated protein was spun at 16000×g for 5 minutes, and the supernatant removed. The protein pellet was dissolved in 100 mM dodecylphosphocholine (DPC) in 50 mM sodium phosphate pH 7.0. The samples were spun for 5 minutes at 16000×g to remove any aggregated protein.

Liposomes were prepared by mixing the appropriate lipids in chloroform and removing the chloroform under vacuum. The dried lipids were resuspended in 1 mL of ddH₂O, vortexed, then extruded through a 0.1 micron membrane to form small unilammelar vesicles. These liposomes were mixed 5:1 with 1.5 mg/mL purified FMS in wash buffer. The sample was sonicated using a Misonix 3000 sonicator equipped with a microtip, then extruded through a 0.1 micron membrane. The liposome samples were dialyzed against 2 L ddH₂O for 12-18 hours with at least 2 changes of the ddH₂O. The liposomes were extruded a second time through a 0.1 micron membrane, and used immediately.

### Characterization

In order to determine the oligimerization state of the FMS peptide in DPC detergent micelles, equilibrium analytical ultracentrifugation was used. To do this, the method of density matching introduced by Tanford and Reynolds was employed (Tanford and Reynolds, Biochim. Biophys. Acta 457; 133-170 (1976), Reynolds and Tanford, Proc. Natl. Acad. Sci. USA 73:4467-4470 (1976)). This method removes the contribution of the detergent to the sedimentation equilibrium data by adjusting the solvent density to be the reciprocal of the partial specific volume of the detergent. The solvent density of the samples was adjusted to this value by adding a final concentration of 58% D₂O to solutions buffered with 50 mM sodium phosphate. The experiments were performed on a Beckman XL-A ultracentrifuge equipped with absorbance optics. The concentration of the protein was monitored at the tryptophan absorbance at 280 nm. Data were collected at 25° C. and three different rotor speeds.

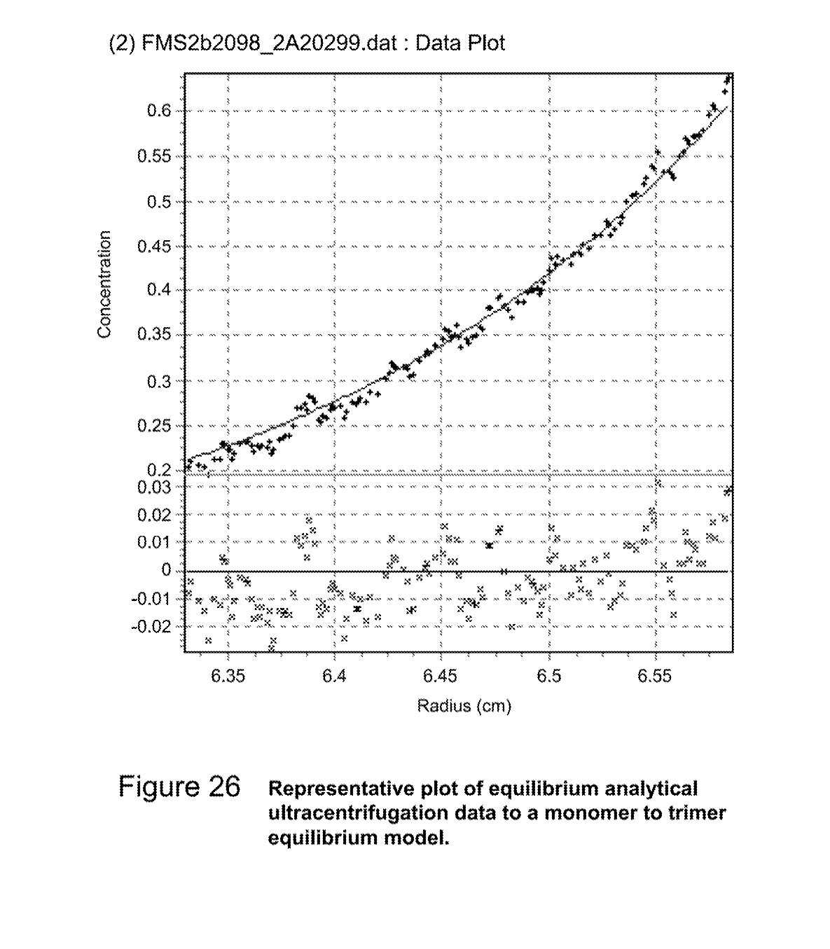

Equilibrium analytical ultracentrifugation has shown that the FMS construct conjugated with micelles is primarily a trimer in solution. Under the conditions used for these experiments, there was a small but detectable population of monomer present in solution, indicating an equilibrium between monomer and trimer. The data could be fit to a monomer to trimer equilibrium model with an equilibrium association constant of 1.12×10¹⁰ M⁻² in 100 mM DPC. Additional data show that the association constant increases very significantly with decreasing detergent concentration. A representative plot of the modeling is shown in FIG. 26. With the experimental conditions used for NMR, the conjugate is almost exclusively a trimer.

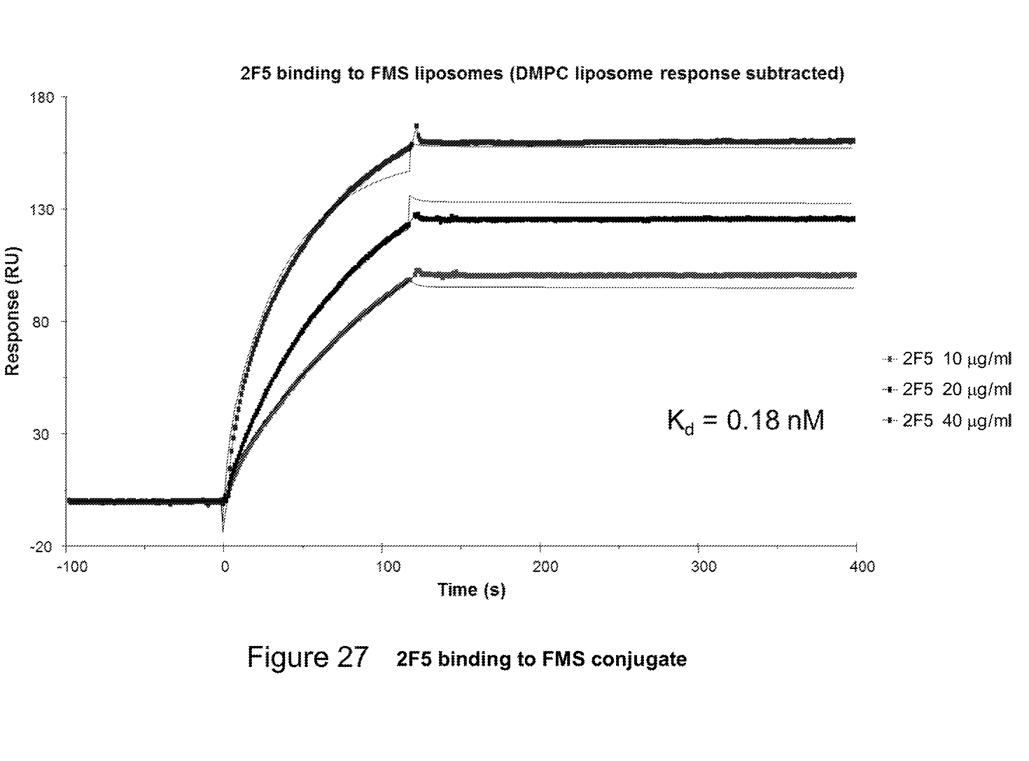

Binding of the FMS conjugate was assayed using surface plasmon resonance. The binding was tested in both liposomes and micelles. In both cases, the FMS conjugate bound well to both 2F5 and 4E10. In DMPC liposomes, the dissociation constants were 0.18 nM and 27 nM for 2F5 and 4E10, respectively. In addition, the FMS conjugate did not interact strongly with the non-neutralizing antibody 13H11 in either DPC micelles or DMPC liposomes. FIG. 27 shows the SPR binding data for 2F5 interacting with the FMS conjugate on DMPC liposomes.

### Example 10

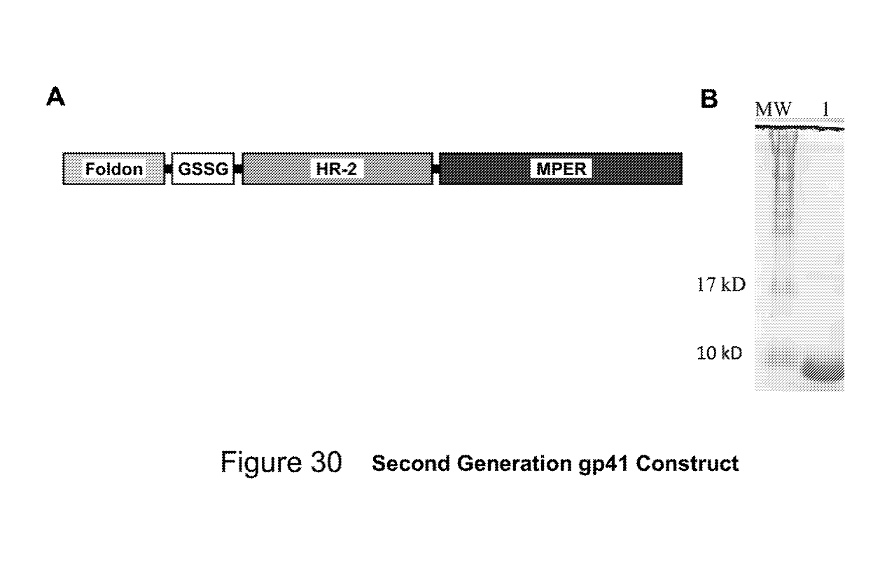

Described below is an extended molecular construct for a trimeric gp41 domain that contains the MPER. This segment of the gp41-M-MAT trimer includes the HR-2 domain shown in FIG. 30. The HR-2 domain plays a critical role in viral infection when it re-folds with HR-1 to form the six-helix bundle that drives membrane fusion. The structure of HR-2 and its corresponding influence on the conformation and immunogenicity of the MPER prior to the formation of the six-helix bundle state has not been previously characterized. This new construct presents the lipid associated MPER and HR-2 domains in a native like pre-fusion intermediate state.

The DNA sequence encoding this peptide was supplied by IDT and inserted into pTCLE. The plasmid pTCLE is a T7 expression vector that contains a modified TrpLE fusion peptide and a 6 histidine-tag (Calderone et al, J. Mol. Biol. 262(4):407-412 (1996); Yansura, Methods Enzymol. 185:161-166 (1990)). As with gp41-M-MAT, this construct expresses well in 041(DE3) E. coli cells and is purified using the same procedure.

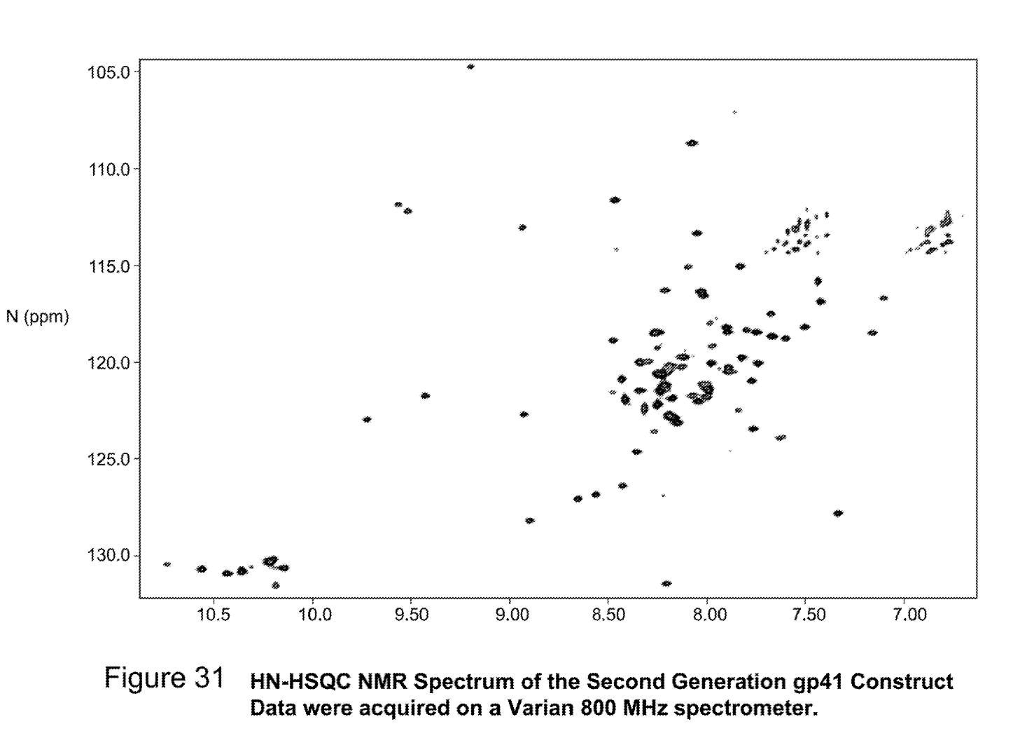

Solution NMR studies of this construct in detergent micelles, show that it yields good NMR spectra as illustrated in FIG. 31. 96% of the backbone resonances are assigned. The NMR spectrum of the foldon domain is identical to that expected for the trimerized state, indicating that the construct is a stable trimer. The backbone amide resonances that correspond to the MPER in this construct are superimposable with those observed for gp41-M-MAT indicating the structure of the MPER domain is nearly identical to that in gp41-M-MAT. Chemical shift indexing indicates that HR-2 forms a core helical region that is separated from the MPER by a flexible segment. Heteronuclear NOE data confirms that the region between the MPER and the HR-2 helix is highly dynamic.

This construct readily associates with DMPC liposomes forming the membrane trimer conjugate. Liposomes were prepared by mixing the appropriate lipids in chloroform and removing the chloroform under vacuum. The dried lipids were resuspended in 1 mL of ddH2O, vortexed, then extruded through a 0.1 micron membrane to form small unilammelar vesicles. These liposomes were mixed 5:1 with 1.5 mg/mL purified trimer in ddH₂O. Buffer was added and the mixture was extruded through a 0.1 micron membrane.

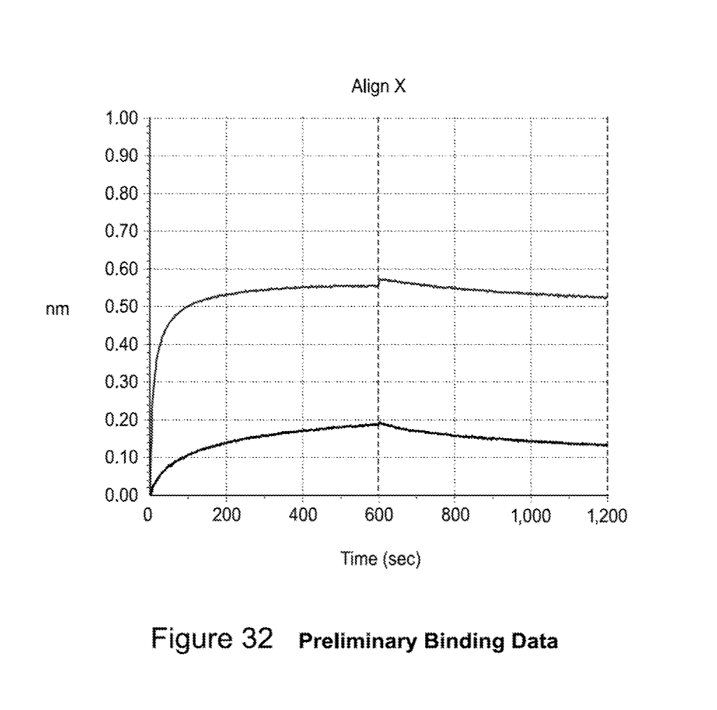

Initial kinetics studies of this construct in DMPC liposomes show that it binds specifically to the neutralizing antibodies, 2F5 and 4E10 (see FIG. 32). This indicates that the 2F5 and 4E10 epitopes are exposed on this molecular trimer structure and the construct is antigenic.

All documents and other information sources cited herein are hereby incorporated in their entirety by reference.

What is claimed is:

1. An immunogenic conjugate comprising a trimerization domain N-terminal to an HIV-1 Env membrane proximal external region (MPER) peptide, wherein said MPER peptide is present as a trimer attached to a biological membrane mimetic.

2. The conjugate according to claim 1 wherein said trimerization domain comprises foldon.

3. The conjugate according to claim 1 wherein said conjugate further comprises a flexible linker between the C-terminus of said trimerization domain and the N-terminus of said MPER peptide.

4. The conjugate according to claim 3 where said linker comprises the peptide GSSG.

5. The conjugate according to claim 1 wherein said conjugate further comprises an HIV-1 HR-2 gp41 domain between the C-terminus of the trimerization domain and the N-terminus of said MPER peptide.

6. A method of inducing an immune response in a subject comprising administering to said subject an amount of said immunogenic conjugate according to claim 1 sufficient to effect said induction.

7. The method according to claim 6 wherein said subject is a human.
